(19) United States
(12) Patent Application Publication
(10) Pub. No.: US 2019/0112674 A1
(43) Pub. Date: Apr. 18, 2019

(54) BIFIDOBACTERIUM ADOLESCENTIS AND USE THEREOF

(71) Applicant: INFINITUS (CHINA) COMPANY LTD., Jiang Men (CN)

(72) Inventors: Yuanyuan WANG, Jiang Men (CN); Wei CHEN, Jiang Men (CN); Gang WANG, Jiang Men (CN); Guangsu ZHU, Jiang Men (CN); Fangli MA, Jiang Men (CN); Lingyun XIAO, Jiang Men (CN); Minghua HU, Jiang Men (CN); Hao ZHANG, Jiang Men (CN); Jianxin ZHAO, Jiang Men (CN); Chung Wah MA, Jiang Men (CN)

(73) Assignee: INFINITUS (CHINA) COMPANY LTD., Jiang Men (CN)

(21) Appl. No.: 16/109,571

(22) Filed: Aug. 22, 2018

(30) Foreign Application Priority Data
Oct. 17, 2017 (CN) 201710963441.5

(51) Int. Cl. C12R 1/01; A61K 35/745; A61P 1/00; A61P 3/00; A23L 33/135

(52) U.S. Cl. CPC C12R 1/01; A61K 35/745; A61K 2035/115; A61P 3/00; A23L 33/135; A61P 1/00

(57) ABSTRACT

Provided is a strain CCFM8630 of Bifidobacterium adolescentis and use thereof. The strain CCFM8630 of Bifidobacterium adolescentis can significantly increase neurotransmitter 5-hydroxytryptamine level in peripheral blood of rat, recover the hormone levels, for example testosterone and so on in peripheral blood of rat, normalize abnormal abundances of Bifidobacterium genus, Blautia genus and Turicibacter genus in intestinal flora of rat affected by high-fat high-starch diet, show pretty good tolerance to simulated gastrointestinal fluid and quickly colonize in intestinal, significantly improve pathological damages of tissues such as liver, duodenum and so on, and increase triglyceride and total cholesterol levels in serum and oral glucose tolerance of rat with metabolic syndrome caused by high-fat high-starch diet. The strain CCFM8630 of Bifidobacterium adolescentis can be used for preventing, relieving or treating metabolic disorder, such as metabolic syndrome, irritable bowel syndrome and mental diseases related to metabolic syndrome such as anxiety, depression and so on.

### CROSS REFERENCE TO RELATED APPLICATIONS

This application claims the priority of Chinese Patent Application No. 201710963441.5, filed on Oct. 17, 2017, and the disclosures of which are hereby incorporated by reference.

### FIELD

The present disclosure relates to the field of microbe technology, specifically to a Bifidobacterium adolescentis and use thereof, especially to a Bifidobacterium adolescentis that is capable of modulating intestinal flora, modulating brain-gut axis and significantly alleviating metabolic syndrome, and use thereof.

### BACKGROUND

Recent years, with the developing economy, life-styles of people in many countries have changed obviously. With the amount of physical activity has decreased, obesity rate has increased significantly, and prevalence rates of diabetes and metabolic syndrome have increased by a large margin. An epidemiology survey shows that 20% to 30% of the adults across the globe are suffering from metabolic syndrome. In 2013, a multicenter, multistage stratified, large-scale sampling survey carried out by Chinese Diabetes Society of Chinese Medical Association showed that among people over 20 years old in large and medium-sized cities, towns and countryside of China, prevalence rates of metabolic syndrome in men and women were 16.7% and 11.7%, respectively, and the total prevalence rate was 13.7%. Furthermore, the rate was continuously increasing. Analyses show that age, blood pressure, diabetes family history, obesity, hyperlipidemia, male, low income and little exercise are main related risk factors for metabolic syndrome. The survey also found that the rates of the overweight and the obesity among people have increased by a large margin, and prevalence rate of metabolic syndrome of male is significant higher than that of female at the same age among middle-aged crowd.

Metabolic syndrome is a clinical syndrome, which has simultaneous symptoms of central obesity, fasting blood glucose rising, high blood pressure, decrease of high-density lipoprotein cholesterol and increase of triacylglycerol, in which numerous hazardous factors basing on the abnormal pathological changes of carbohydrate metabolism, lipid metabolism and protein metabolism aggregates, and which promotes development of diabetes (type II) and cardiovascular diseases such as atherosclerosis and so on. Due to metabolic syndrome is a pathological condition in which numerous metabolic abnormalities aggregates, its occurrence is relates to insulin resistance, becoming a hot spot in the research field of cardiovascular diseases and diabetes, and raising many controversies at home and abroad. In addition, metabolic syndrome accompanies with disorder of intestinal microecosystem. Disorder of intestinal microecosystem may further lead to disorder of intestinal functions, nerve center functions and peripheral nerve functions, for example, intestinal inflammation, inflammatory bowel disease (IBD) and irritable bowel syndrome (IBS), abnormality of neurotransmitter 5-hydroxytryptamine level and level of some hormone and so on. Many researches have shown that abundances of intestinal microbes of some genus have intimate connection with intestinal diseases, for example, the abundance of Blautia genus will rise in intestinal flora of IBS patient. At the same time, researches also show that mental diseases such as depression, anxiety and so on have intimate connection with metabolic syndrome, intestinal flora disorder, and low 5-hydroxytryptamine level in human body. Improving 5-hydroxytryptamine level in peripheral blood helps increasing neurotransmitter level of central nervous system, so that relieving symptoms of anxiety, depression and so on.

At present, all the drug treatments of metabolic syndrome aim to decrease all kinds of risk factors, and these drugs include: anti-obesity drugs for weight loss; dimethylbiguanide and thiazolidinediones for reducing insulin resistance; sulfonylurea and rosiglitazone for controlling blood glucose; fibrate and statin for improving disorder of lipid metabolism; captopril, amlodipine and so on for controlling the blood pressure; drugs for treating intestinal diseases such as IBD, IBS and so on, including glucocorticoid, immunosuppressant, psychotropic drugs and so on; drugs for mental diseases such as anxiety, depression and so on, including selective serotonin reuptake inhibitors such as paroxetine, noradrenaline, and specific 5-hydroxytryptamine antidepressant drugs such as mirtazapine and so on. All of the medicines above have certain therapeutic effects, but as the conditions getting worse, the amounts of medicine used increase, the interactions between the medicines as well as the toxic and side effects of medicines become significant, leading to adverse reaction of digestive tract and showing liver and renal toxicity in some degree. In consideration of problems of the medicines, early intervention in metabolic syndrome, intestinal disease and mental disease can effectively decrease onset risk of cardiovascular and cerebrovascular diseases, diabetes, inflammatory bowel diseases, depression and so on.

Probiotics are edible microbes that are beneficial to human health, which have potential functions of alleviating abnormal metabolism of blood glucose and blood lipid, and modulating intestinal flora proportion and brain-gut axis. Thus, there is important social and economy value to research and develop probiotic products that can effectively intervene the occurrence and development of metabolic syndrome, intestinal diseases and mental diseases.

At present, there is no patent about using probiotics to increase 5-hydroxytryptamine level so as to regulate brain-gut axis and relieve anxiety and depression. Although there is related patent application (CN107083339A) that discloses adding Blautia bacteria to protect piglets from diarrhea, there is no patent about modulating the abundance of Blautia genus bacteria in intestinal tract so as to alleviate intestinal disease by the uptake of edible microbes (list of bacterium that can be permitted to be used in food, infant food, health products by National Health Commission of the People's Republic of China, 2014). In addition, there are some patents or patent application relating to compositions and preparation method thereof for preventing and curing metabolic syndrome. For example, CN104906263A discloses a composition consisting of tea polyphenol, procyanidin and POTENTILLAE DISCOLORIS HERBA extract, which is used to treat metabolic syndrome. CN105796674A discloses a traditional Chinese medicine composition comprising PLANTAGINIS SEMEN, COPTIDIS RHIZOMA and so on, which is capable of preventing and curing metabolic syndrome. In addition, a few patents relate to probiotics-containing compositions that are used to improve metabolic syndrome, and the methods for preparing the same. For example, CN105816623A discloses a probiotic-fermented traditional Chinese medicine composition being used to cure and improve metabolic syndrome, which is made from traditional Chinese medicine such as PANACIS QUINQUEFOLII RADIX, DIOSCOREAE RHIZOMA, MOUTAN CORTEX, PORIA by extracting and fermenting the extract with probiotics. All the above patents and patent applications are using traditional Chinese medicine components or a mixture of bacteria and traditional Chinese medicine to alleviate metabolic syndrome, in which the bacteria and the functions of the bacteria are not clear. CN105567586A discloses a Lactobacillus plantarum NCU116 with antidiabetic function, which achieves antidiabetic effects through modulating body blood glucose, blood lipid, hormone level and body metabolism. The Lactobacillus plantarum is screened and selected from bacteria in kimchi instead of human sources. No evidence shows that Lactobacillus plantarum can colonize in human intestinal tract and take effects. So far, there is no a human-sourced individual probiotic (such as Bifidobacterium) that can colonize in human intestinal tract to relieve the metabolic syndrome, or to alleviate symptoms such as hyperglycemia, hyperlipidemia, intestinal flora imbalance, intestinal inflammation and so on, and related mental diseases.

### SUMMARY

In view of above, an object of the present disclosure is to solve the problems in the prior art by providing a probiotics. The probiotics can colonize in intestinal tract of human body, improve 5-hydroxytryptamine level in peripheral blood, regulate brain-gut axis and recovering testosterone level in serum back to normal level, normalize abnormal abundances of Blautia genus and Turicibacter genus in intestinal flora, improve metabolic syndrome, relieve hyperglycemia, hyperlipidemia and inflammation of liver and duodenum, liver fibrosis and other symptoms.

The present disclosure provides a strain CCFM8630 of Bifidobacterium adolescentis, which is deposited at China General Microbiological Culture Collection Center (CGMCC, Address: Beijing Institute of Microbiology, Chinese Academy of Sciences, No. 1, Beichen West Road, Chaoyang District, Beijing, China) on Jul. 7, 2017, with an accession number CGMCC 14395.

In one embodiment, the present disclosure studies effect of strain CCFM8630 of Bifidobacterium adolescentis on intestinal flora imbalance caused by high-carbohydrate and high-fat diet. The results show that the uptake of strain CCFM8630 of Bifidobacterium adolescentis significantly recovers relative abundances of Bifidobacterium genus, Turicibacter genus and Blautia genus in disordered intestinal microbes of rat feces, and the intervention effect is obviously better than that of Bifidobacterium animalis BB12.

In one embodiment, the present disclosure studies protection effects of strain CCFM8630 of Bifidobacterium adolescentis on tissue damages of liver, duodenum and so on in rat with metabolic syndrome. The results show that administration of strain CCFM8630 of Bifidobacterium adolescentis by intragastric gavage significantly improves symptoms caused by high-fat high-starch diet, such as hepatocyte microvesicular steatosis, interstitial inflammatory cell infiltration, early fibrosis of liver tissue, duodenum villi broadening, interstitial edema, increasing of inflammatory cells and so on in rats, and the intervention effect is obviously better than that of Bifidobacterium animalis BB12.

In one embodiment, the present disclosure studies effect of strain CCFM8630 of Bifidobacterium adolescentis on (fasting) blood glucose level of rat with metabolic syndrome. The results show that administration of strain CCFM8630 of Bifidobacterium adolescentis by intragastric gavage obviously decreases the fasting blood glucose level of the model rat close to the blank control group. The ability of strain CCFM8630 of Bifidobacterium adolescentis on decreasing fasting blood glucose level of rat is better than that of rosiglitazone and Bifidobacterium animalis BB12 by intragastric gavage administration.

In one embodiment, the present disclosure studies effect of strain CCFM8630 of Bifidobacterium adolescentis on oral glucose tolerance of rat with metabolic syndrome. The results show that strain CCFM8630 of Bifidobacterium adolescentis significantly improves oral glucose tolerance and the effect is better than that of Bifidobacterium animalis BB12, indicating that strain CCFM8630 of Bifidobacterium adolescentis can further decrease glucose level by improving glucose tolerance.

In one embodiment, the present disclosure studies effects of strain CCFM8630 of Bifidobacterium adolescentis on total cholesterol (TC) and triglyceride (TG) in serum of rat with metabolic syndrome, respectively. The results show that administration of strain CCFM8630 of Bifidobacterium adolescentis by intragastric gavage decreases levels of total cholesterol and triglyceride in serum.

Further, in one embodiment, the present disclosure studies effects of strain CCFM8630 of Bifidobacterium adolescentis on 5-HT and testosterone level in serum of rat with metabolic syndrome, respectively. The results show that administration of strain CCFM8630 of Bifidobacterium adolescentis by intragastric gavage improves 5-hydroxytryptamine (5-HT) level in rat serum, and reduces testosterone in serum back to normal level.

Therefore, the present disclosure provides use of strain CCFM8630 of Bifidobacterium adolescentis in preparing products that can improve metabolic syndrome, regulate intestinal flora or regulate brain-gut axis.

Therein, the improving metabolic syndrome is to relieve symptoms of hyperglycemia and hyperlipidemia, inflammation of liver and duodenum, and liver tissue fibrosis; the modulating intestinal flora is to normalize abnormal abundances of Blautia genus and Turicibacter genus in intestinal flora; and the modulating brain-gut axis as well as relieving anxiety and depression is to increase 5-hydroxytryptamine level in peripheral blood.

The product of the present disclosure includes but is not limited to health food or pharmaceutical preparation.

Therein, the health food includes but is not limited to microbial agent or fermented food.

Further, the present disclosure provides a microbial agent comprising the strain CCFM8630 of Bifidobacterium adolescentis.

Preferably, the viable count of the strain CCFM8630 of Bifidobacterium adolescentis in the microbial agent is more than 10⁶ CFU/g

The microbial agent of the present disclosure can be prepared by routine methods.

In some embodiments, the method for preparing the microbial agent is:

inoculating the strain CCFM8630 of Bifidobacterium adolescentis to a modified MRS medium at an inoculum size of 2 to 4 wt %, culturing for 24 to 48h at a temperature between 35 and 39° C. under anaerobic conditions, collecting bacteria, resuspending the bacteria with a protectant to reach a bacterial density of 10¹⁰ CFU/mL, culturing the suspension at 37° C. for 50 to 70 minutes under anaerobic conditions, and drying the resulting culture.

Therein, the modified MRS medium (mMRS) in the present disclosure is a MRS medium containing 0.05% of L-cysteine hydrochloride. The specific method for preparing the medium is: dissolving 10 g of tryptone, 10 g of beef extract, 5 g of yeast powder, 20 g of glucose, 5 g of sodium acetate, 2 g of ammonium citrate dibasic, 2 g of dipotassium hydrogen phosphate, 0.5 g of magnesium sulfate heptahydrate, 1 mL of Tween-80, 0.25 g of manganese sulfate monohydrate and 0.5 g of cysteine hydrochloride in water, diluting the mixture to 1000 mL with water, modulating the pH to 6.5, and autoclaving at 119-123° C. for 15 to 25 minutes.

The protectant in the method of the present disclosure is an aqueous solution that contains 100 g/L to 150 g/L of nonfat milk powder, 100 g/L to 150 g/L of maltodextrin and 140 g/L to 160 g/L of trehalose. That is, the protectant consists of nonfat milk powder, maltodextrin, trehalose and water, wherein the concentration of nonfat milk powder is from 100 g/L to 150 g/, the concentration of maltodextrin is from 100 g/L to 150 g/L, and the concentration of trehalose is from 140 g/L to 160 g/L.

Preferably, in the method of the present disclosure, bacteria collected after culturing in the modified MRS medium are subjected to washing with phosphate buffer solution for 2 to 4 times, and pH of the phosphate buffer solution is from 6.8 to 7.2.

The drying of the method in the present disclosure can be any of the drying procedures for bacteria solution, for example vacuum freeze-drying. In some embodiments, the drying of the present disclosure is vacuum freeze-drying after pre-freezing the bacteria at −15 to −20° C. for 8 to 14h.

The present disclosure also provides a fermented food which is produced by using the strain CCFM8630 of Bifidobacterium adolescentis as a starter culture.

The fermented food is fermented dairy products, fermented bean products or fermented fruit and vegetable products.

The fermented dairy products include but are not limited to yogurt, sour cream and cheese. The fermented bean products include but are not limited to soymilk, fermented beans and bean paste. The fruits and vegetables in the fermented fruit and vegetable products include but are not limited to cucumber, carrot, beet, celery and cabbage.

The present disclosure also provides a pharmaceutical preparation, comprising an effective amount of the strain CCFM8630 of Bifidobacterium adolescentis and a pharmaceutically acceptable adjuvant.

The pharmaceutically acceptable adjuvant is one or more selected from the group consisting of filler, adhesive, wetting agent, disintegrating agent, lubricant, and flavoring agent.

In some embodiments of the present disclosure, the pharmaceutical preparation is a granule, a capsule, a tablet, a pill or an oral liquid.

The beneficial technical effects of the present disclosure are as follows.

The strain CCFM8630 of Bifidobacterium adolescentis of the present disclosure significantly increases neurotransmitter 5-hydroxytryptamine (5-HT) level in peripheral blood of rat, regulates brain-gut axis, relieves mental illnesses related to metabolic syndrome, for example anxiety, depression and so on, recovers the hormone level, for example testosterone and so on in peripheral blood of rat caused by high-fat high-starch diet, recovers abundances of Bifidobacterium genus, Blautia genus and Turicibacter genus in abnormal intestinal flora of rat caused by high-fat high-starch diet. In addition, strain CCFM8630 of Bifidobacterium adolescentis has pretty good tolerance to simulated gastrointestinal fluid, and quickly colonizes in intestinal, significantly alleviates pathology damages of tissues, such as liver, duodenum and so on of rat with metabolic syndrome caused by high-fat high-starch diet; significantly improves oral glucose tolerance of rat with metabolic syndrome and decreases the under curve area of glucose tolerance test; significantly increases triglyceride and total cholesterol levels in serum of rat with metabolic syndrome caused by high-fat high-starch diet. The strain CCFM8630 of Bifidobacterium adolescentis of the present disclosure can be used to prepare health foods or medicines that improve metabolic syndrome, regulates intestinal flora, relieves irritable bowel syndrome, regulates brain-gut axis and alleviates mental illness such as anxiety, depression and so on, which has a pretty wide application prospect.

### Description of Microbiological Preservation

CCFM8630, classification name: Bifidobacterium adolescentis, is deposited at China General Microbiological Culture Collection Center (CGMCC, Address: Beijing Institute of Microbiology, Chinese Academy of Sciences, No. 1, Beichen West Road, Chaoyang District, Beijing, China) on Jul. 7, 2017, with an accession number CGMCC 14395.

### BRIEF DESCRIPTION OF DRAWINGS

In order to describe the technical solutions in the examples of the present disclosure or the conventional art more clearly, the accompanying drawings used in description of the embodiments or the prior art will be illustrated briefly.

FIG. 1 shows colony morphology of strain CCFM8630 of Bifidobacterium adolescentis.

FIG. 2 shows effects of strain CCFM8630 of Bifidobacterium adolescentis on tissue structure of liver in rat with metabolic syndrome.

FIG. 3 shows effects of strain CCFM8630 of Bifidobacterium adolescentis on tissue structure of duodenum in rat with metabolic syndrome.

FIG. 4 shows effects of strain CCFM8630 of Bifidobacterium adolescentis on abundances of some intestinal microbes in rat with metabolic syndrome; and there are significant differences (P<0.05) between a, b and c groups.

FIG. 5 shows effects of strain CCFM8630 of Bifidobacterium adolescentis on oral glucose tolerance in rat with metabolic syndrome; and there are significant differences (P<0.05) between a, b and c groups.

FIG. 6 is a curve showing changes of blood glucose level in oral glucose tolerance test.

FIG. 7 shows the area under the curve (AUC_{glucose}) in oral glucose tolerance test.

FIG. 8 shows effects of strain CCFM8630 of Bifidobacterium adolescentis on total cholesterol (TC) in serum of rat with metabolic syndrome; and there are significant differences (P<0.05) between a, b and c groups.

FIG. 9 shows effects of strain CCFM8630 of Bifidobacterium adolescentis on triglyceride (TG) in serum of rat with metabolic syndrome; and there are significant differences (P<0.05) between a, b and c groups.

FIG. 10 shows effects of strain CCFM8630 of Bifidobacterium adolescentis on 5-hydroxytryptamine (5-HT) and testosterone levels of rat with metabolic syndrome; and there are significant differences (P<0.05) between a, b and c groups.

### DETAILED DESCRIPTION

The present disclosure discloses a Bifidobacterium adolescentis and use thereof. One of ordinary skill in the art can learn from the contents herein and improve the process parameters appropriately. In particular, it shall be noted that all the similar substitutions and modifications are apparent to one of ordinary skill in the art and are to be considered within the scope of the present invention. The method and product of the present invention have been described with preferred examples. It is apparent that one of the ordinary skill in the art can make change or modify the combination to the method and product of the present invention without departing from the spirit, scope and spirit of the invention, therefore realizing and applying the techniques of the present invention. www

The strain CCFM8630 of Bifidobacterium adolescentis of the present disclosure has the following biology properties.

(1) Bacterium properties: Gram staining positive, without spore, not moving

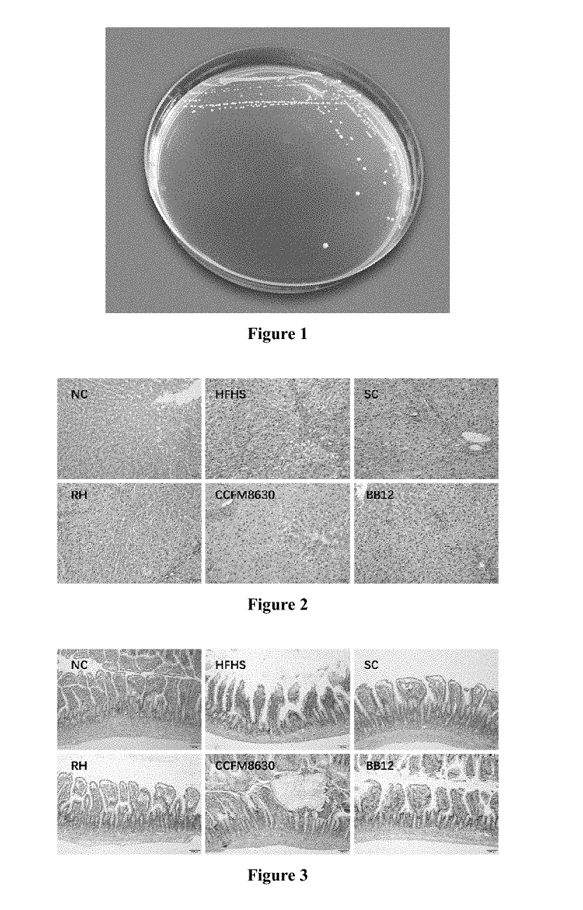

(2) Colony properties: colonies are formed after 36-hour anaerobic culture, the diameters of the colonies are between 0.5 and 2 mm, the front view is a circle and the side view is a protuberance, and the edge is smooth, the color is milky and non-transparent, the surface is moist and smooth, and no pigment is produce. See FIG. 1.

(3) Growth properties: the bacteria are cultured in mMRS medium under anaerobic condition for about 24-hour at constant temperature of 37° C. to reach log phase.

(4) Good tolerance to simulate gastrointestinal fluid.

(5) Significantly improve pathological tissue damages such as liver, duodenum and so on of rat with from metabolic syndrome.

(6) Significantly improve oral glucose tolerance of rat with metabolic syndrome.

(7) Decrease area under the curve in glucose tolerance test.

(8) Regulate the levels of triglyceride and total cholesterol in serum back to normal level.

(9) Increase 5-hydroxytryptamine (5-HT) level in peripheral blood and regulate testosterone to normal level.

(10) Significantly recover abundances of Bifidobacterium genus, Turicibacter genus, Blautia genus and so on in abnormal intestinal flora caused by high-fat high-starch diet.

The strain CCFM8630 of Bifidobacterium adolescentis of the present disclosure is obtained by the following method.

### I. Isolation and Screening of Lactobacillus

(1) 1 g of fresh feces was diluted in gradient, spreaded on solid mMRS medium, and cultured at 37° C. for 72 hours under anaerobic condition. The feces were obtained from a 95-year-old male from Changshou Village, Chaihu Town in Zhongxiang City of Hubei Province, China.

(2) Morphology of the colonies were observed and recorded, and single colony was picked out and purified by streaking.

(3) The bacteria were cultured at 37° C. for 48 hours in mMRS medium, and the colonies obtained were subjected to Gram Staining. The morphologies of the colonies were recorded.

(4) The Gram-negative strains and Gram-positive cocci were discarded, the Gram-positive bacilli were selected.

(5) The bacteria were subjected to catalase analyzing, the catalase-positive strains were discarded and catalase-negative strains were retained.

### II. Preliminary Identification of Bifidobacterium: Fructose-6-Phosphoketolase Assay

(1) The Lactobacillus obtained in Step I was cultured in mMRS liquid medium for 24 hours, and then 1 mL of the culture was taken and centrifuged at 8000 rpm for 2 minutes.

(2) The bacterial pellet was washed two times with 0.05M KH₂PO₄ solution (pH 6.5) containing 0.05% (mass percentage) of cysteine hydrochloride.

(3) The bacteria was resuspended in 200 μL of the phosphate buffer above with an addition of 0.25% (mass percentage) Triton X-100.

(4) 50 μL mixture solution of 6 mg/mL sodium fluoride and 10 mg/mL sodium iodoacetate, and 50 μL of 80 mg/mL fructose-6-phosphoric acid were added and incubated at 37° C. for 1 hour.

(5) 300 μL of 0.139 g/mL hydroxylamine hydrochloride solution (pH 6.5) was added and placed at room temperature for 10 minutes.

(6) 200 μL of 15% (mass percentage) trichloroacetic acid and 200 μL of 4M HCl were added, respectively.

(7) 200 μL of 0.1M HCl containing 5% (mass percentage) ferric chloride was added. The color of the system turned red quickly, indicating a F6PPK-positive reaction. Therefore, the bacteria were initially identified as Bifidobacterium.

### III. Molecular Biological Identification of Bifidobacterium

(1) Genome extraction of single bacterium (according to operation procedures of TIANamp Bacteria DNA kit)

A. The Lactobacillus obtained in Step II was cultured overnight. 1 mL culture was put into a 1.5 mL centrifuge tube and centrifuged at 10,000 rpm (˜11,500×g) for 1 minute. The supernatant was removed as much as possible.

B. 180 μL buffer (20 mg/mL lysozyme solution with 20 mM Tris (pH 8.0), 2 mM Na₂-EDTA, and 1.2% Triton) was added to the bacteria and incubated at 37° C. for more than 30 minutes. (The lysozyme solution should be prepared by dissolving lysozyme dry powder in the buffer, or the lysozyme would be inactive.

C. 20 μL Proteinase K solution was added to the tube and mixed well.

D. 220 μL buffer GB was added, shaken for 15 seconds, and placed at 70° C. for 10 minutes. The solution turned clean. The tube was centrifuged for a few seconds to remove water drops on inner wall of the tube.

E. 220 μL absolute alcohol was added adequately shaken for 15 seconds. Flocculent precipitates maybe appear. The tube was centrifuged for a few seconds to remove water drops on inner wall of the tube.

F. The solution and flocculent precipitate obtained in the last step were put into an adsorption column CB3 (the absorption column was disposed in a collecting tube), and subjected to centrifugation at 12,000 rpm (˜13,400×g) for 30 seconds. The flow-through liquor was discarded, and the adsorption column was put back into the collecting tube.

G. 500 μL of buffer GD (check for absolute alcohol adding before use) was added to the adsorption column CB3. The column was centrifuged at 12,000 rpm (˜13,400×g) for 30 seconds. The flow-through liquor was discarded, and the adsorption column was put back into the collecting tube.

H. 600 μL of washing solution PW (check for absolute alcohol adding before use) was added to the adsorption column CB3. The column was centrifuged at 12,000 rpm (˜13,400×g) for 30 seconds. The flow-through liquor was discarded, and the adsorption column was put back into the collecting tube. This step was repeated once.

I. The adsorption column CB3 was put back into the collecting tube, centrifuged at 12,000 rpm (˜13,400×g) for 2 minutes, and the flow-through liquor was discarded. The adsorption column CB3 was placed at room temperature for a few minutes to let the adsorption column totally dry.

J. The adsorption column CB3 was transferred to a clean centrifugal tube, and 50 to 200 μL of elution buffer TE was dropped to the middle of the adsorption film. The adsorption column was placed at room temperature for 2 to 5 minutes, and then centrifuged at 12,000 rpm (˜13,400×g) for 2 minutes. The eluted solution was collected into a centrifuge tube.

(2) Whole Genome Sequencing

The extracted whole genome was sent to a professional sequencing company and a second-generation sequencer was used to sequence the whole bacterial genome. The sequencing results were subjected to similarity comparison by BLAST software among GeneBank database. The results show that the strain provided by the present disclosure is a Bifidobacterium adolescentis belonging to Bifidobacterium genus, but different from known Bifidobacterium adolescentis, so that it is identified as a new strain. By blastn algorithm, the genome of strain CCFM8630 of Bifidobacterium adolescentis was compared with that of standard Bifidobacterium adolescentis strain ATCC15703 (https://www.ncbi.nlm.nih.gov/genome/?term=683), 259 genes in total show differences, which are shown in Table 1.

TABLE 1
Differential genes between strain CCFM8630 of Bifidobacterium adolescentis
and standard strain ATCC15703 of Bifidobacterium adolescentis

| | | | | Correlation |
|---|---|---|---|---|
| Gene ID | Nr Database Article ID | Note of the Result | Similarity | Length |
| Z25_GM000009 | gi\|748204304\|ref\|WP_039774992.1\| | hypothetical protein[Bifidobacterium | 76.423 | 123 |
| | | adolescentis] | | |
| Z25_GM000013 | gi\|822625123\|ref\|WP_046999178.1\| | hypothetical protein [Bifidobacterium | 87.5 | 128 |
| | | adolescentis] | | |
| Z25_GM000014 | gi\|822625124\|ref\|WP_046999179.1\| | transcriptional regulator | 95.062 | 81 |
| | | [Bifidobacterium adolescentis] | | |
| Z25_GM000019 | gi\|748204310\| ref\|WP_039774998.1\| | hypothetical protein [Bifidobacterium | 100 | 81 |
| | | adolescentis] | | |
| Z25_GM000024 | gi\|748204315\|ref\|WP_039775003.1\| | single-stranded DNA-binding | 81.921 | 177 |
| | | protein [Bifidobacterium | | |
| | | adolescentis] | | |
| Z25_GM000026 | gi\|748204318\|ref\|WP_039775006.1\| | hypothetical protein [Bifidobacterium | 88.889 | 63 |
| | | adolescentis] | | |
| Z25_GM000029 | gi\|748204322\|ref\|WP_039775010.11 | hypothetical protein [Bifidobacterium | 35 | 80 |
| | | adolescentis] | | |
| Z25_GM000031 | gi\|673003250\|gb\|KFI98296.1\| | hypothetical protein BSTER_0878 | 50.909 | 55 |
| | | [Bifidobacterium stercoris JCM 15918] | | |
| Z25_GM000032 | gi\|154085053\|gb\|EDN84098.1\| | hypothetical protein BIFADO_01031 | 54.545 | 44 |
| | | [Bifidobacterium adolescentis L2-32] | | |
| Z25_GM000034 | gi\|748204324\|ref\|WP_039775012.1\| | hypothetical protein [Bifidobacterium | 89.787 | 235 |
| | | adolescentis] | | |
| Z25_GM000035 | gi\|748204325\|ref\|WP_039775013.1\| | HNH endonuclease [Bifidobacterium | 94.495 | 109 |
| | | adolescentis] | | |
| Z25_GM000038 | gi\|917738554\|ref\|WP_052252740.1\| | hypothetical protein [Bifidobacterium | 34.874 | 476 |
| | | adolescentis] | | |
| Z25_GM000039 | gi\|917738475\|ref\|WP_052252661.1\| | hypothetical protein [Bifidobacterium | 43.805 | 226 |
| | | adolescentis] | | |
| Z25_GM000047 | gi\|917738481\|ref\|WP_052252667.1\| | hypothetical protein [Bifidobacterium | 100 | 206 |
| | | adolescentis] | | |
| Z25_GM000048 | gi\|748204335\|ref\|WP_039775023.1\| | hypothetical protein [Bifidobacterium | 97.619 | 126 |
| | | adolescentis] | | |
| Z25_GM000049 | gi\|748204337\|ref\|WP_039775025.1\| | hypothetical protein [Bifidobacterium | 100 | 66 |
| | | adolescentis] | | |
| Z25_GM000053 | gi\|747124343\|gb\|AJE05765.1\| | Phage tail fiber protein | 91.367 | 139 |
| | | [Bifidobacterium adolescentis] | | |
| Z25_GM000053 | gi\|747124343\|gb\|AJE05765.1\| | Phage tail fiber protein | 85.185 | 54 |
| | | [Bifidobacterium adolescentis] | | |
| Z25_GM000054 | gi\|917738485\|ref\|WP_052252671.1\| | hypothetical protein [Bifidobacterium | 60.938 | 192 |
| | | adolescentis] | | |
| Z25_GM000058 | gi\|917738487\|ref\|WP_052252673.1\| | hypothetical protein [Bifidobacterium | 94.318 | 88 |
| | | adolescentis] | | |
| Z25_GM000059 | gi\|747124349\|gb\|AJE05771.1\| | Integrase [Bifidobacterium | 99.251 | 267 |
| | | adolescentis] | | |
| Z25_GM000079 | gi\|489906072\|ref\|WP_003809496.1\| | hypothetical protein [Bifidobacterium | 100 | 78 |
| | | adolescentis] | | |
| Z25_GM000121 | gi\|747124414\|gb\|AJE05836.1\| | excinuclease subunit A [Bifidobacterium | 100 | 768 |
| | | adolescentis] | | |
| Z25_GM000122 | gi\|489905980\|ref\|WP_003809404.1\| | MULTISPECIES: GNAT family | 100 | 137 |
| | | N-acetyltransferase [Bifidobacterium] | | |
| Z25_GM000123 | gi\|705407595\|ref\|WP_033499438.1\| | alpha/beta hydrolase [Bifidobacterium | 99.7 | 333 |
| | | adolescentis] | | |
| Z25_GM000155 | gi\|154083897\|gb\|EDN82942.1\| | hypothetical protein BIFADO_01229 | 100 | 125 |
| | | [Bifidobacterium adolescentis L2-32] | | |
| Z25_GM000158 | gi\|500063247\|ref\|WP_011743164.1\| | hypothetical protein [Bifidobacterium | 100 | 502 |
| | | adolescentis] | | |
| Z25_GM000206 | gi\|489905824\|ref\|WP_003809249.1\| | transcriptional regulator [Bifidobacterium | 100 | 198 |
| | | adolescentis] | | |
| Z25_GM000208 | gi\|737015357\|ref\|WP_035010987.1\| | hypothetical protein [Bifidobacterium | 100 | 49 |
| | | adolescentis] | | |
| Z25_GM000214 | gi\|489905803\|ref\|WP_003809228.1\| | heat-inducible transcriptional | 100 | 381 |
| | | repressor HrcA [Bifidobacterium | | |
| | | adolescentis] | | |
| Z25_GM000236 | gi\|673003360\|gb\|KFI98406.1\| | hypothetical protein BSTER_6020 | 100 | 36 |
| | | [Bifidobacterium stercoris | | |
| | | JCM 15918] | | |
| Z25_GM000237 | gi\|748204479\| ref\|WP_039775167.1\| | hypothetical protein [Bifidobacterium | 100 | 84 |
| | | adolescentis] | | |
| Z25_GM000262 | gi\|500063319\| ref\|WP_011743236.1\| | DNA-deoxyinosine glycosylase | 99.441 | 179 |
| | | [Bifidobacterium adolescentis] | | |
| Z25_GM000266 | gi\|747124562\| gb\|AJE05984.1\| | hypothetical protein BBMN23_1012 | 100 | 38 |
| | | [Bifidobacterium adolescentis] | | |
| Z25_GM000267 | gi\|757771530\|ref\|WP_042991197.1\| | hypothetical protein [Bifidobacterium | 100 | 71 |
| | | adolescentis] | | |
| Z25_GM000273 | gi\|740659212\|ref\|WP_038444512.1\| | hypothetical protein [Bifidobacterium | 100 | 112 |
| | | adolescentis] | | |
| Z25_GM000279 | gi\|118765497\|dbj\|BAF39676.1\| | transcriptional regulator [Bifidobacterium | 100 | 92 |
| | | adolescentis ATCC 15703] | | |
| Z25_GM000289 | gi\|489931370\|ref\|WP_003834689.1\| | MULTISPECIES: cell division | 100 | 294 |
| | | protein Fic [Bifidobacterium] | | |
| Z25_GM000297 | gi\|489906825\|ref\|WP_003810247.1\| | hypothetical protein [Bifidobacterium | 100 | 910 |
| | | adolescentis] | | |
| Z25_GM000298 | gi\|489906826\|ref\|WP_003810248.1\| | hypothetical protein [Bifidobacterium | 100 | 329 |
| | | adolescentis] | | |
| Z25_GM000300 | gi\|154082753\|gb\|EDN81798.1\| | DNA (cytosine-5-)-methyltransferase | 100 | 234 |
| | | [Bifidobacterium adolescentis L2-32] | | |
| Z25_GM000302 | gi\|489906833\|ref\|WP_003810255.1\| | hypothetical protein [Bifidobacterium | 99.852 | 674 |
| | | adolescentis] | | |
| Z25_GM000303 | gi\|740659235\|ref\|WP_038444535.1\| | hypothetical protein [Bifidobacterium | 99.342 | 152 |
| | | adolescentis] | | |
| Z25_GM000304 | gi\|154082758\|gb\|EDN81803.1\| | hypothetical protein BIFADO_01928 | 100 | 33 |
| | | [Bifidobacterium adolescentis L2-32] | | |
| Z25_GM000306 | gi\|154082760\|gb\|EDN81805.1\| | hypothetical protein BIFADO_01930 | 100 | 42 |
| | | [Bifidobacterium adolescentis L2-32] | | |
| Z25_GM000308 | gi\|154082763\|gb\|EDN81808.1\| | hypothetical protein BIFADO_01933 | 81.395 | 43 |
| | | [Bifidobacterium adolescentis L2-32] | | |
| Z25_GM000313 | gi\|705409744\|ref\|WP_033500282.1\| | NAD(+) kinase [Bifidobacterium | 98.726 | 314 |
| | | adolescentis] | | |
| Z25_GM000321 | gi\|673003127\|gb\|KFI98173.1\| | transporter [Bifidobacterium | 100 | 243 |
| | | stercoris JCM 15918] | | |
| Z25_GM000322 | gi\|673003126\|gb\|KFI98172.1\| | HD superfamily metal-dependent | 99.408 | 169 |
| | | phosphohydrolase [Bifidobacterium | | |
| | | stercoris JCM 15918] | | |
| Z25_GM000325 | gi\|489906866\|ref\|WP_003810288.1\| | arginine repressor [Bifidobacterium | 100 | 172 |
| | | adolescentis] | | |
| Z25_GM000331 | gi\|489906872\|ref\|WP_003810294.1\| | hypothetical protein [Bifidobacterium | 97.357 | 227 |
| | | adolescentis] | | |
| Z25_GM000332 | gi\|740659259\|ref\|WP_038444559.1\| | phenylalanine-tRNA ligase subunit | 99.654 | 867 |
| | | beta [Bifidobacterium adolescentis] | | |
| Z25_GM000341 | gi\|489906895\|ref\|WP_003810317.1\| | membrane protein [Bifidobacterium | 100 | 262 |
| | | adolescentis] | | |
| Z25_GM000345 | gi\|489906905\|ref\|WP_003810327.1\| | cobalt ABC transporter permease | 100 | 292 |
| | | [Bifidobacterium adolescentis] | | |
| Z25_GM000356 | gi\|747124649\|gb\|AJE06071.1\| | putative glycosyltransferase | 100 | 360 |
| | | [Bifidobacterium adolescentis] | | |
| Z25_GM000361 | gi\|671342920\|gb\|IAII76436.1\| | helicase [Bifidobacterium | 99.603 | 1261 |
| | | adolescentis] | | |
| Z25_GM000364 | gi\|154082824\|gb\|EDN81869.1\| | Ion channel [Bifidobacterium | 100 | 241 |
| | | adolescentis L2-32] | | |
| Z25_GM000373 | gi\|917265320\|ref\|WP_051872032.1\| | hypothetical protein [Bifidobacterium | 100 | 248 |
| | | adolescentis] | | |
| Z25_GM000375 | gi\|751368899\|gb\|KIM01425.1\| | hypothetical protein LU08_05925 | 100 | 61 |
| | | [Bifidobacterium adolescentis] | | |
| Z25_GM000380 | gi\|118765587\|dbj\|BAF39766.1\| | putative DNA polymerase III | 99.517 | 207 |
| | | epsilon subunit [Bifidobacterium | | |
| | | adolescentis ATCC 15703] | | |
| Z25_GM000381 | gi\|751368903\|gb\|KIM01429.1\| | homocysteine methyltransferase | 100 | 294 |
| | | [Bifidobacterium adolescentis] | | |
| Z25_GM000383 | gi\|747124679\|gb\|AJE06101.1\| | amino acid transport protein | 100 | 109 |
| | | [Bifidobacterium adolescentis] | | |
| Z25_GM000390 | gi\|118765595\|dbj\|BAF39774.1\| | hypothetical protein BAD_0993 | 98.551 | 69 |
| | | [Bifidobacterium adolescentis | | |
| | | ATCC 15703] | | |
| Z25_GM000401 | gi\|118765606\|dbj\|BAF39785.1\| | hypothetical protein BAD_1004 | 100 | 103 |
| | | [Bifidobacterium adolescentis | | |
| | | ATCC 15703] | | |
| Z25_GM000415 | gi\|154082938\|gb\|EDN81983.1\| | hypothetical protein BIFADO_02108 | 100 | 43 |
| | | [Bifidobacterium adolescentis L2-32] | | |
| Z25_GM000420 | gi\|489907169\|ref\|WP_003810591.1\| | recombination regulator RecX | 100 | 231 |
| | | [Bifidobacterium adolescentis] | | |
| Z25_GM000421 | gi\|489907170\|ref\|WP_003810592.1\| | DNA recombination/repair protein | 100 | 337 |
| | | RecA [Bifidobacterium adolescentis] | | |
| Z25_GM000423 | gi\|489907175\|ref\|WP_003810597.1\| | transcriptional regulator [Bifidobacterium | 100 | 169 |
| | | adolescentis] | | |
| Z25_GM000432 | gi\|740659333\|ref\|WP_038444633.1\| | membrane protein [Bifidobacterium | 100 | 324 |
| | | adolescentis] | | |
| Z25_GM000464 | gi\|547082934\|ref\|WP_021913854.1\| | hypothetical protein [Bifidobacterium | 100 | 72 |
| | | adolescentis] | | |
| Z25_GM000470 | gi\|747124764\|gb\|AJE06186.1\| | hypothetical protein BBMN23_1214 | 100 | 267 |
| | | [Bifidobacterium adolescentis] | | |
| Z25_GM000472 | gi\|489907274\|ref\|WP_003810696.1\| | site-specific tyrosine recombinase | 100 | 317 |
| | | XerD [Bifidobacterium | | |
| | | adolescentis] | | |
| Z25_GM000474 | gi\|489907277\|ref\|WP_003810699.1\| | MULTISPECIES: 50S ribosomal | 100 | 64 |
| | | protein L35 [Bifidobacterium] | | |
| Z25_GM000482 | gi\|489907294\|ref\|WP_003810716.1\| | hypothetical protein [Bifidobacterium | 100 | 79 |
| | | adolescentis] | | |
| Z25_GM000496 | gi\|118765699\|dbj\|BAF39878.1\| | DEAD/DEAH box helicase-like | 95.745 | 47 |
| | | [Bifidobacterium adolescentis | | |
| | | ATCC 15703] | | |
| Z25_GM000506 | gi\|705408403\|ref\|WP_033499726.1\| | cell division protein [Bifidobacterium | 99.833 | 600 |
| | | adolescentis] | | |
| Z25_GM000509 | gi\|547083061\|ref\|WP_021913877.1\| | division/cell wall cluster tran- | 100 | 171 |
| | | scriptional repressor MraZ | | |
| | | [Bifidobacterium adolescentis] | | |
| Z25_GM000510 | gi\|740659382\|ref\|WP_038444682.1\| | hypothetical protein [Bifidobacterium | 98.462 | 65 |
| | | adolescentis] | | |
| Z25_GM000513 | gi\|489907353\|ref\|WP_003810775.1\| | transcriptional regulator NrdR | 100 | 150 |
| | | [Bifidobacterium adolescentis] | | |
| Z25_GM000514 | gi\|751368419\|gb\|KIM00984.1\| | peptidoglycan-binding protein | 100 | 85 |
| | | [Bifidobacterium adolescentis] | | |
| Z25_GM000561 | gi\|489904214\|ref\|WP_003807643.1\| | MULTISPECIES: nucleotidyltrans- | 100 | 263 |
| | | ferase [Bifidobacterium] | | |
| Z25_GM000577 | gi\|736988102\|ref\|WP_034984157.1\| | hypothetical protein [Bifidobacterium | 95.683 | 278 |
| | | adolescentis] | | |
| Z25_GM000579 | gi\|751369378\|gb\|KIM01887.1\| | hypothetical protein LU08_03085 | 100 | 59 |
| | | [Bifidobacterium adolescentis] | | |
| Z25_GM000580 | gi\|657871024\|ref\|WP_029575615.1\| | MULTISPECIES: polyketide | 95 | 60 |
| | | cyclase [Terrabacteria group] | | |
| Z25_GM000581 | gi\|490750313\|ref\|WP_004612621.1\| | MULTISPECIES: competence | 100 | 74 |
| | | protein TfoX [Firmicutes] | | |
| Z25_GM000582 | gi\|154084484\|gb\|EDN83529.1\| | hypothetical protein BIFADO_00438 | 100 | 262 |
| | | [Bifidobacterium adolescentis L2-32] | | |
| Z25_GM000583 | gi\|154084485\|gb\|EDN83530.1\| | hypothetical protein BIFADO_00439 | 100 | 48 |
| | | [Bifidobacterium adolescentis L2-32] | | |
| Z25_GM000590 | gi\|489904267\|ref\|WP_003807696.1\| | 50S ribosomal protein L32 | 100 | 64 |
| | | [Bifidobacterium adolescentis] | | |
| Z25_GM000655 | gi\|154084571\|gb\|EDN83616.1\| | hypothetical protein BIFADO_00529 | 97.778 | 90 |
| | | [Bifidobacterium adolescentis L2-32] | | |
| Z25_GM000657 | gi\|489904451\|ref\|WP_003807880.1\| | FmdB family transcriptional regulator | 98.333 | 60 |
| | | [Bifidobacterium adolescentis] | | |
| Z25_GM000667 | gi\|748204088\|ref\|WP_039774776.1\| | hypothetical protein [Bifidobacterium | 96.639 | 119 |
| | | adolescentis] | | |
| Z25_GM000668 | gi\|740658926\|ref\|WP_038444227.1\| | ABC transporter substrate-binding | 30.162 | 431 |
| | | protein [Bifidobacterium adolescentis] | | |
| Z25_GM000669 | gi\|736880360\|ref\|WP_034879947.1\| | peptide ABC transporter permease | 98.485 | 330 |
| | | [Bifidobacterium pseudocatenulatum] | | |
| Z25_GM000670 | gi\|736880694\|ref\|WP_034880276.1\| | ABC transporter permease | 100 | 191 |
| | | [Bifidobacterium pseudocatenulatum] | | |
| Z25_GM000671 | gi\|705411392\|ref\|WP_033500823.1\| | ABC transporter ATP-binding | 77.061 | 279 |
| | | protein [Bifidobacterium | | |
| | | kashiwanohense] | | |
| Z25_GM000752 | gi\|740658820\|ref\|WP_038444121.1\| | oleate hydratase [Bifidobacterium | 99.681 | 626 |
| | | adolescentis] | | |
| Z25_GM000764 | gi\|489906187\|ref\|WP_003809611.1\| | DoxX [Bifidobacterium adolescentis] | 95.181 | 83 |
| Z25_GM000770 | gi\|705409239\|ref\|WP_033500070.1\| | hypothetical protein [Bifidobacterium | 93.443 | 122 |
| | | adolescentis] | | |
| Z25_GM000771 | gi\|736508496\|ref\|WP_034524549.1\| | hypothetical protein [Bifidobacterium | 91.971 | 137 |
| | | adolescentis] | | |
| Z25_GM000772 | gi\|673000940\|gb\|KFI96007.1\| | hypothetical protein BSTER_1719 | 91.071 | 56 |
| | | [Bifidobacterium stercoris | | |
| | | JCM 15918] | | |
| Z25_GM000774 | gi\|489905678\|ref\|WP_003809103.1\| | nitrate reductase [Bifidobacterium | 81.865 | 193 |
| | | adolescentis] | | |
| Z25_GM000776 | gi\|705409248\|ref\|WP_033500073.1\| | hypothetical protein [Bifidobacterium | 68.121 | 298 |
| | | adolescentis] | | |
| Z25_GM000780 | gi\|489906219\|ref\|WP_003809643.1\| | hypothetical protein [Bifidobacterium | 41.791 | 201 |
| | | adolescentis] | | |
| Z25_GM000782 | gi\|705409263\|ref\|WP_033500080.1\| | hypothetical protein [Bifidobacterium | 69.663 | 89 |
| | | adolescentis] | | |
| Z25_GM000783 | gi\|705409266\|ref\|WP_033500081.1\| | hypothetical protein [Bifidobacterium | 58.333 | 132 |
| | | adolescentis] | | |
| Z25_GM000784 | gi\|705409268\|ref\|WP_033500082.1\| | hypothetical protein [Bifidobacterium | 99.415 | 171 |
| | | adolescentis] | | |
| Z25_GM000789 | gi\|705409278\|ref\|WP_033500087.1\| | terminase [Bifidobacterium | 100 | 537 |
| | | adolescentis] | | |
| Z25_GM000791 | gi\|705409284\|ref\|WP_033500090.1\| | PhnA protein [Bifidobacterium | 97.083 | 240 |
| | | adolescentis] | | |
| Z25_GM000793 | gi\|705409286\|ref\|WP_033500091.1\| | GTP-binding protein | 90.385 | 52 |
| | | [Bifidobacterium adolescentis] | | |
| Z25_GM000795 | gi\|154084106\|gb\|EDN83151.1\| | hypothetical protein BIFADO_00046 | 92.982 | 57 |
| | | [Bifidobacterium adolescentis L2-32] | | |
| Z25_GM000796 | gi\|673000966\|gb\|KFI96033.1\| | hypothetical protein BSTER_1745 | 90.909 | 44 |
| | | [Bifidobacterium stercoris | | |
| | | JCM 15918] | | |
| Z25_GM000798 | gi\|705409296\|ref\|WP_033500096.1\| | hypothetical protein [Bifidobacterium | 97.619 | 84 |
| | | adolescentis] | | |
| Z25_GM000799 | gi\|705409298\|ref\|WP_033500097.1\| | hypothetical protein [Bifidobacterium | 91.176 | 68 |
| | | adolescentis] | | |
| Z25_GM000800 | gi\|489905582\|ref\|WP_003809007.1\| | methyltransferase [Bifidobacterium | 100 | 161 |
| | | adolescentis] | | |
| Z25_GM000801 | gi\|705409306\|ref\|WP_033500101.1\| | hypothetical protein [Bifidobacterium | 100 | 125 |
| | | adolescentis] | | |
| Z25_GM000802 | gi\|673000975\|gb\|KFI96042.1\| | putative ferredoxin [Bifidobacterium | 98.148 | 54 |
| | | stercoris JCM 15918] | | |
| Z25_GM000808 | gi\|489905562\|ref\|WP_003808987.1\| | hypothetical protein [Bifidobacterium | 83.051 | 59 |
| | | adolescentis] | | |
| Z25_GM000809 | gi\|705409319\|ref\|WP_033500107.1\| | hypothetical protein [Bifidobacterium | 91.429 | 70 |
| | | adolescentis] | | |
| Z25_GM000810 | gi\|705409321\|ref\|WP_033500108.1\| | antirepressor [Bifidobacterium | 60.902 | 266 |
| | | adolescentis] | | |
| Z25_GM000811 | gi\|489903579\|ref\|WP_003807009.1\| | hypothetical protein [Bifidobacterium | 77 | 200 |
| | | adolescentis] | | |
| Z25_GM000815 | gi\|705409328\|ref\|WP_033500111.11 | hypothetical protein [Bifidobacterium | 97.333 | 75 |
| | | adolescentis] | | |
| Z25_GM000817 | gi\|489905540\|ref\|WP_003808965.1\| | anhydro-N-acetylmuramyl-tripep- | 90.476 | 42 |
| | | tide amidase [Bifidobacterium | | |
| | | adolescentis] | | |
| Z25_GM000818 | gi\|489905537\|ref\|WP_003808962.1\| | transcriptional regulator [Bifidobacterium | 93.333 | 120 |
| | | adolescentis] | | |
| Z25_GM000822 | gi\|917316857\|ref\|WP_051923569.1\| | hypothetical protein [Bifidobacterium | 31.288 | 163 |
| | | adolescentis] | | |
| Z25_GM000834 | gi\|154085026\|gb\|EDN84071.1\| | hypothetical protein BIFADO_01003 | 100 | 28 |
| | | [Bifidobacterium adolescentis L2-32] | | |
| Z25_GM000835 | gi\|489905505\|ref\|WP_003808930.1\| | general stress protein | 100 | 171 |
| | | [Bifidobacterium adolescentis] | | |
| Z25_GM000856 | gi\|747124227\|gb\|AJE05649.1\| | Type I restriction-modification | 40.845 | 213 |
| | | system specificity subunit S | | |
| | | [Bifidobacterium adolescentis] | | |
| Z25_GM000856 | gi\|747124227\|gb\|AJE05649.1\| | Type I restriction-modification | 31.313 | 198 |
| | | system specificity subunit S | | |
| | | [Bifidobacterium adolescentis] | | |
| Z25_GM000858 | gi\|737014132\|ref\|WP_035009793.1\| | integrase [Bifidobacterium | 99.675 | 308 |
| | | adolescentis] | | |
| Z25_GM000870 | gi\|154084979\|gb\|EDN84024.1\| | hypothetical protein BIFADO_00956 | 96.078 | 102 |
| | | [Bifidobacterium adolescentis L2-32] | | |
| Z25_GM000873 | gi\|154084979\|gb\|EDN84024.1\| | hypothetical protein BIFADO_00956 | 76.19 | 105 |
| | | [Bifidobacterium adolescentis L2-32] | | |
| Z25_GM000874 | gi\|154084978\|gb\|EDN84023.1\| | hypothetical protein BIFADO_00955 | 90.476 | 63 |
| | | [Bifidobacterium adolescentis L2-32] | | |
| Z25_GM000881 | gi\|489905358\|ref\|WP_003808783.1\| | histidine kinase [Bifidobacterium | 98.619 | 869 |
| | | adolescentis] | | |
| Z25_GM000915 | gi\|154084913\|gb\|EDN83958.1\| | universal stress family protein | 100 | 335 |
| | | [Bifidobacterium adolescentis L2-32] | | |
| Z25_GM000921 | gi\|489905270\|ref\|WP_003808696.1\| | type VII secretion protein | 100 | 96 |
| | | [Bifidobacterium adolescentis] | | |
| Z25_GM000926 | gi\|489905257\|ref\|WP_003808683.1\| | hypothetical protein [Bifidobacterium | 100 | 317 |
| | | adolescentis] | | |
| Z25_GM000927 | gi\|489905255\|ref\|WP_003808681.11 | hypothetical protein [Bifidobacterium | 100 | 174 |
| | | adolescentis] | | |
| Z25_GM000928 | gi\|489905253\|ref\|WP_003808679.1\| | VWA domain-containing protein | 99.718 | 355 |
| | | [Bifidobacterium adolescentis] | | |
| Z25_GM000930 | gi\|489905249\|ref\|WP_003808675.1\| | cell surface protein [Bifidobacterium | 99.459 | 185 |
| | | adolescentis] | | |
| Z25_GM000932 | gi\|489905244\|ref\|WP_003808670.1\| | hypothetical protein [Bifidobacterium | 99.229 | 519 |
| | | adolescentis] | | |
| Z25_GM000943 | gi\|500063070\|ref\|WP_011742987.1\| | phosphoserine phosphatase SerB | 100 | 227 |
| | | [Bifidobacterium adolescentis] | | |
| Z25_GM000966 | gi\|489905157\|ref\|WP_003808583.1\| | hypothetical protein [Bifidobacterium | 100 | 75 |
| | | adolescentis] | | |
| Z25_GM001016 | gi\|751369644\|gb\|KIM02148.1\| | fimbrial protein [Bifidobacterium | 83.333 | 534 |
| | | adolescentis] | | |
| Z25_GM001017 | gi\|748205409\|ref\|WP_039776097.11 | hypothetical protein [Bifidobacterium | 100 | 184 |
| | | adolescentis] | | |
| Z25_GM001020 | gi\|671342004\|gb\|AII75520.1\| | fimbriae protein with LPXTG | 98.077 | 728 |
| | | motif and von Willebrand factor | | |
| | | typeA domain [Bifidobacterium | | |
| | | adolescentis] | | |
| Z25_GM001042 | gi\|673001223\|gb\|KFI96287.1\| | TetR-type transcriptional regulator | 100 | 225 |
| | | [Bifidobacterium stercoris | | |
| | | JCM 15918] | | |
| Z25_GM001043 | gi\|705408020\|ref\|WP_033499573.11 | phage infection protein [Bifidobacterium | 99.863 | 731 |
| | | adolescentis] | | |
| Z25_GM001046 | gi\|1489903990\|ref\|WP_003807419.1\| | NrdH-redoxin [Bifidobacterium | 100 | 78 |
| | | adolescentis] | | |
| Z25_GM001051 | gi\|154084351\|gb\|EDN83396.1\| | hypothetical protein BIFADO_00303 | 98.246 | 57 |
| | | [Bifidobacterium adolescentis L2-32] | | |
| Z25_GM001067 | gi\|489933463\|ref\|WP_003836777.1\| | MULTISPECIES: ABC transporter | 100 | 268 |
| | | permease [Bifidobacterium] | | |
| Z25_GM001068 | gi\|748203972\|ref\|WP_039774660.1\| | hypothetical protein [Bifidobacterium | 99.205 | 629 |
| | | adolescentis] | | |
| Z25_GM001069 | gi\|747123689\|gb\|AJE05111.1\| | Hypothetical protein BBMN23_0139 | 98.467 | 1566 |
| | | [Bifidobacterium adolescentis] | | |
| Z25_GM001073 | gi\|748203974\|ref\|WP_039774662.1\| | hypothetical protein [Bifidobacterium | 99.225 | 258 |
| | | adolescentis] | | |
| Z25_GM001149 | gi\|154084733\|gb\|EDN83778.1\| | hypothetical protein BIFADO_00702 | 100 | 123 |
| | | [Bifidobacterium adolescentis L2-32] | | |
| Z25_GM001157 | gi\|489904938\|ref\|WP_003808365.1\| | acetylesterase [Bifidobacterium | 100 | 272 |
| | | adolescentis] | | |
| Z25_GM001178 | gi\|489904982\|ref\|WP_003808409.1\| | peptidase Ml3 [Bifidobacterium | 100 | 696 |
| | | adolescentis] | | |
| Z25_GM001194 | gi\|747124065\|gb\|AJE05487.1\| | hypothetical protein BBMN23_0515 | 97.674 | 43 |
| | | [Bifidobacterium adolescentis] | | |
| Z25_GM001220 | gi\|747125053\|gb\|AJE06475.1\| | putative transport protein | 98.879 | 535 |
| | | [Bifidobacterium adolescentis] | | |
| Z25_GM001244 | gi\|489906423\|ref\|WP_003809846.1\| | hypothetical protein [Bifidobacterium | 100 | 332 |
| | | adolescentis] | | |
| Z25_GM001245 | gi\|705409011\|ref\|WP_033499974.1\| | hypothetical protein [Bifidobacterium | 100 | 363 |
| | | adolescentis] | | |
| Z25_GM001248 | gi\|651887219\|ref\|WP_026646861.1\| | ATP synthase F0F1 subunit | 100 | 92 |
| | | epsilon [Bifidobacterium | | |
| | | ruminantium] | | |
| Z25_GM001252 | gi\|489906439\|ref\|WP_003809862.1\| | ATP synthase subunit delta | 100 | 275 |
| | | [Bifidobacterium adolescentis] | | |
| Z25_GM001266 | gi\|748205152\|ref\|WP_039775840.1\| | hypothetical protein [Bifidobacterium | 100 | 643 |
| | | adolescentis] | | |
| Z25_GM001268 | gi\|740659818\|ref\|WP_038445118.1\| | AsnC family transcriptional | 100 | 158 |
| | | regulator [Bifidobacterium | | |
| | | adolescentis] | | |
| Z25_GM001270 | gi\|154083598\|gb\|EDN82643.1\| | hypothetical protein BIFADO_01696 | 96.774 | 93 |
| | | [Bifidobacterium adolescentis L2-32] | | |
| Z25_GM001276 | gi\|500063830\|ref\|WP_011743747.1\| | aspartyl/glutamyl-tRNA(Asn/Gln) | 100 | 98 |
| | | amidotransferase subunit C | | |
| | | [Bifidobacterium adolescentis] | | |
| Z25_GM001282 | gi\|489906654\|ref\|WP_003810077.1\| | 2-hydroxyhepta-2,4-diene-1,7-dioate | 99.634 | 273 |
| | | isomerase [Bifidobacterium | | |
| | | adolescentis] | | |
| Z25_GM001286 | gi\|547062388\|ref\|WP_021912974.1\| | YggS family pyridoxal | 100 | 272 |
| | | phosphate enzyme | | |
| | | [Bifidobacterium adolescentis] | | |
| Z25_GM001301 | gi\|917316766\|ref\|WP_051923478.1\| | hypothetical protein [Bifidobacterium | 38.806 | 67 |
| | | adolescentis] | | |
| Z25_GM001321 | gi\|154084201\|gb\|EDN83246.1\| | 16S rRNA methyltransferase GidB | 100 | 247 |
| | | [Bifidobacterium adolescentis L2-32] | | |
| Z25_GM001341 | gi\|489903773\|ref\|WP_003807203.1\| | hypothetical protein [Bifidobacterium | 100 | 155 |
| | | adolescentis] | | |
| Z25_GM001366 | gi\|747123591\|gb\|AJE05013.1\| | hypothetical protein BBMN23_0041 | 100 | 49 |
| | | [Bifidobacterium adolescentis] | | |
| Z25_GM001377 | gi\|822624998\|ref\|WP_046999053.1\| | hypothetical protein [Bifidobacterium | 99.715 | 351 |
| | | adolescentis] | | |
| Z25_GM001381 | gi\|671341961\|gb\|AII75477.1\| | putative membrane protein | 98.708 | 774 |
| | | [Bifidobacterium adolescentis] | | |
| Z25_GM001382 | gi\|500062698\|ref\|WP_011742615.1\| | ABC transporter [Bifidobacterium | 98.893 | 903 |
| | | adolescentis] | | |
| Z25_GM001387 | gi\|154083139\|gb\|EDN82184.1\| | hypothetical protein BIFADO_02312 | 100 | 42 |
| | | [Bifidobacterium adolescentis L2-32] | | |
| Z25_GM001396 | gi\|748204836\|ref\|WP_039775524.1\| | phosphoesterase [Bifidobacterium | 100 | 268 |
| | | adolescentis] | | |
| Z25_GM001397 | gi\|489907517\|ref\|WP_003810939.1\| | ATP-binding protein | 100 | 74 |
| | | [Bifidobacterium adolescentis] | | |
| Z25_GM001403 | gi\|705408881\|ref\|WP_033499925.1\| | cell division protein DivIVA | 98.473 | 524 |
| | | [Bifidobacterium adolescentis] | | |
| Z25_GM001406 | gi\|154083118\|gb\|EDN82163.1\| | hypothetical protein BIFADO_02290 | 100 | 541 |
| | | [Bifidobacterium adolescentis L2-32] | | |
| Z25_GM001421 | gi\|154083101\|gb\|EDN82146.1\| | hypothetical protein BIFADO_02273 | 98.889 | 90 |
| | | [Bifidobacterium adolescentis L2-32] | | |
| Z25_GM001422 | gi\|822625273\|ref\|WP_046999328.1\| | phosphate ABC transporter | 99.519 | 208 |
| | | [Bifidobacterium adolescentis] | | |
| Z25_GM001426 | gi\|748204805\|ref\|WP_039775493.1\| | hypothetical protein [Bifidobacterium | 99.667 | 601 |
| | | adolescentis] | | |
| Z25_GM001427 | gi\|489907456\|ref\|WP_003810878.1\| | type II secretion system | 100 | 591 |
| | | protein E [Bifidobacterium | | |
| | | adolescentis] | | |
| Z25_GM001428 | gi\|489907454\|ref\|WP_003810876.1\| | prepilin-type N-terminal | 100 | 264 |
| | | cleavage/methylation domain- | | |
| | | containing protein [Bifidobacterium | | |
| | | adolescentis] | | |
| Z25_GM001429 | gi\|822582229\|gb\|KLE27724.1\| | hypothetical protein AAX71_05000 | 100 | 191 |
| | | [Bifidobacterium adolescentis] | | |
| Z25_GM001432 | gi\|917738515\|ref\|WP_052252701.1\| | hypothetical protein [Bifidobacterium | 100 | 805 |
| | | adolescentis] | | |
| Z25_GM001436 | gi\|489907436\|ref\|WP_003810858.1\| | hypothetical protein [Bifidobacterium | 100 | 203 |
| | | adolescentis] | | |
| Z25_GM001441 | gi\|500063552\|ref\|WP_011743469.1\| | hemolysin [Bifidobacterium | 98.63 | 73 |
| | | adolescentis] | | |
| Z25_GM001446 | gi\|118765731\|dbj\|BAF39910.1\| | histidinol dehydrogenase | 100 | 27 |
| | | [Bifidobacterium adolescentis ATCC | | |
| | | 15703] | | |
| Z25_GM001449 | gi\|547074020\|ref\|WP_021913401.1\| | imidazoleglycerol-phosphate | 100 | 199 |
| | | dehydratase [Bifidobacterium | | |
| | | adolescentis] | | |
| Z25_GM001456 | gi\|740659385\|ref\|WP_038444685.1\| | hypothetical protein [Bifidobacterium | 98.895 | 181 |
| | | adolescentis] | | |
| Z25_GM001457 | gi\|751368642\|gb\|KIM01183.1\| | hypothetical protein LU08_07305 | 84.651 | 215 |
| | | [Bifidobacterium adolescentis] | | |
| Z25_GM001458 | gi\|747124820\|gb\|AJE06242.1\| | hypothetical protein BBMN23_1270 | 90 | 90 |
| | | [Bifidobacterium adolescentis] | | |
| Z25_GM001460 | gi\|671343072\|gb\|AII76588.1\| | hypothetical protein BADO_1174 | 91.892 | 185 |
| | | [Bifidobacterium adolescentis] | | |
| Z25_GM001467 | gi\|489907882\|ref\|WP_003811303.1\| | hypothetical protein [Bifidobacterium | 100 | 83 |
| | | adolescentis] | | |
| Z25_GM001481 | gi\|489907849\|ref\|WP_003811270.1\| | sugar ABC transporter permease | 100 | 299 |
| | | [Bifidobacterium adolescentis] | | |
| Z25_GM001483 | gi\|154083302\|gb\|EDN82347.1\| | haloacid dehalogenase-like hydrolase | 99.598 | 249 |
| | | [Bifidobacterium adolescentis L2-32] | | |
| Z25_GM001489 | gi\|737015814\|ref\|WP_035011431.1\| | adhesin [Bifidobacterium | 98.382 | 309 |
| | | adolescentis] | | |
| Z25_GM001496 | gi\|705409435\|ref\|WP_033500154.1\| | MerR family transcriptional regulator | 100 | 178 |
| | | [Bifidobacterium adolescentis] | | |
| Z25_GM001497 | gi\|651887462\|ref\|WP_026647098.1\| | MULTISPECIES: hypothetical | 100 | 55 |
| | | protein [Bifidobacterium] | | |
| Z25_GM001499 | gi\|651887464\|ref\|WP_026647100.11 | MULTISPECIES: ATPase | 99.758 | 413 |
| | | [Bifidobacterium] | | |
| Z25_GM001508 | gi\|748205242\|ref\|WP_039775930.1\| | alpha-xylosidase | 100 | 804 |
| | | [Bifidobacterium adolescentis] | | |
| Z25_GM001520 | gi\|154083261\|gb\|EDN82306.1\| | hypothetical protein BIFADO_02438 | 97.059 | 34 |
| | | [Bifidobacterium adolescentis L2-32] | | |
| Z25_GM001532 | gi\|154083241\|gb\|EDN82286.1\| | hypothetical protein BIFADO_02416 | 100 | 69 |
| | | [Bifidobacterium adolescentis L2-32] | | |
| Z25_GM001533 | gi\|500063643\|ref\|WP_011743560.1\| | alpha-mannosidase | 99.579 | 949 |
| | | [Bifidobacterium adolescentis] | | |
| Z25_GM001536 | gi\|500063640\|ref\|WP_011743557.1\| | membrane protein | 100 | 149 |
| | | [Bifidobacterium adolescentis] | | |
| Z25_GM001540 | gi\|500063637\|ref\|WP_011743554.1\| | DNA mismatch repair protein | 21.366 | 454 |
| | | MutH [Bifidobacterium | | |
| | | adolescentis] | | |
| Z25_GM001557 | gi\|154083218\|gb\|EDN82263.1\| | anaerobic ribonucleoside-triphos- | 100 | 237 |
| | | phate reductase activating | | |
| | | protein [Bifidobacterium | | |
| | | adolescentis L2-32] | | |
| Z25_GM001559 | gi\|671343544\|gb\|AII77060.1\| | glutamate-cysteine ligase | 100 | 423 |
| | | [Bifidobacterium adolescentis] | | |
| Z25_GM001567 | gi\|740659868\|ref\|WP_038445168.1\| | AMP-binding protein | 29.651 | 172 |
| | | [Bifidobacterium adolescentis] | | |
| Z25_GM001569 | gi\|154083208\|gb\|EDN82253.1\| | hypothetical protein BIFADO_02381 | 100 | 27 |
| | | [Bifidobacterium adolescentis L2-32] | | |
| Z25_GM001573 | gi\|489907668\|ref\|WP_003811090.1\| | AcrR family transcriptional regulator | 100 | 211 |
| | | [Bifidobacterium adolescentis] | | |
| Z25_GM001581 | gi\|705408834\|ref\|WP_033499905.1\| | MarR family transcriptional regulator | 99.561 | 228 |
| | | [Bifidobacterium adolescentis] | | |
| Z25_GM001595 | gi\|154083714\|gb\|EDN82759.1\| | dCTP deaminase | 99.512 | 205 |
| | | [Bifidobacterium adolescentis L2-32] | | |
| Z25_GM001609 | gi\|747125322\|gb\|AJE06744.1\| | hypothetical protein BBMN23_1772 | 97.368 | 38 |
| | | [Bifidobacterium adolescentis] | | |
| Z25_GM001611 | gi\|736987863\|ref\|WP_034983920.1\| | hypothetical protein [Bifidobacterium | 99.676 | 309 |
| | | adolescentis] | | |
| Z25_GM001612 | gi\|751369357\|gb\|KIM01867.1\| | MFS transporter [Bifidobacterium | 98.446 | 193 |
| | | adolescentis] | | |
| Z25_GM001613 | gi\|489903120\|ref\|WP_003806552.1\| | hypothetical protein [Bifidobacterium | 97.214 | 323 |
| | | adolescentis] | | |
| Z25_GM001647 | gi\|747124986\|gb\|AJE06408.1\| | hypothetical protein BBMN23_1436 | 99.432 | 176 |
| | | [Bifidobacterium adolescentis] | | |
| Z25_GM001648 | gi\|705409118\|ref\|WP_033500018.1\| | hypothetical protein [Bifidobacterium | 98.905 | 274 |
| | | adolescentis] | | |
| Z25_GM001649 | gi\|705409855\|ref\|WP_033500321.1\| | glycosyl transferase family 2 | 99.543 | 657 |
| | | [Bifidobacterium adolescentis] | | |
| Z25_GM001651 | gi\|705409864\|ref\|WP_033500324.1\| | sugar ABC transporter | 79.621 | 422 |
| | | [Bifidobacterium adolescentis] | | |
| Z25_GM001652 | gi\|705409866\|ref\|WP_033500325.1\| | glycosyl transferase family 9 | 88.129 | 278 |
| | | [Bifidobacterium adolescentis] | | |
| Z25_GM001653 | gi\|705409873\|ref\|WP_033500328.1\| | galactofuranosyltransferase | 89.338 | 619 |
| | | [Bifidobacterium adolescentis] | | |
| Z25_GM001655 | gi\|751368680\|gb\|KIM01218.1\| | deoxyribonuclease [Bifidobacterium | 60.656 | 61 |
| | | adolescentis] | | |
| Z25_GM001658 | gi\|705409861\|ref\|WP_033500323.1\| | hypothetical protein [Bifidobacterium | 34.84 | 376 |
| | | adolescentis] | | |
| Z25_GM001660 | gi\|705409873\|ref\|WP_033500328.1\| | galactofuranosyltransferase | 31.613 | 620 |
| | | [Bifidobacterium adolescentis] | | |
| Z25_GM001662 | gi\|705409861\|ref\|WP_033500323.1\| | hypothetical protein [Bifidobacterium | 32.68 | 153 |
| | | adolescentis] | | |
| Z25_GM001664 | gi\|751369058\|gb\|KIM01578.1\| | glycosyl transferase [Bifidobacterium | 88.818 | 313 |
| | | adolescentis] | | |
| Z25_GM001665 | gi\|737015663\|ref\|WP_035011282.1\| | hypothetical protein [Bifidobacterium | 28.689 | 366 |
| | | adolescentis] | | |
| Z25_GM001677 | gi\|489903795\|ref\|WP_003807225.1\| | hypothetical protein [Bifidobacterium | 56.559 | 709 |
| | | adolescentis] | | |
| Z25_GM001678 | gi\|747124812\|gb\|AJE06234.1\| | L-lactate dehydrogenase 2 | 32.646 | 291 |
| | | [Bifidobacterium adolescentis] | | |
| Z25_GM001686 | gi\|154084014\|gb\|EDN83059.1\| | ABC 3 transport family protein | 100 | 280 |
| | | [Bifidobacterium adolescentis L2-32] | | |
| Z25_GM001691 | gi\|500063170\|ref\|WP_011743087.1\| | membrane protein [Bifidobacterium | 100 | 330 |
| | | adolescentis] | | |
| Z25_GM001694 | gi\|747124275\|gb\|AJE05697.1\| | hypothetical protein BBMN23_0725 | 100 | 106 |
| | | [Bifidobacterium adolescentis] | | |
| Z25_GM001698 | gi\|914800850\|ref\|WP_050731476.1\| | transcriptional regulator | 99.794 | 486 |
| | | [Bifidobacterium adolescentis] | | |
| Z25_GM001702 | gi\|489906165\|ref\|WP_003809589.1\| | MULTISPECIES: WhiB family | 100 | 71 |
| | | transcriptional regulator | | |
| | | [Bifidobacterium] | | |
| Z25_GM001710 | gi\|489903317\|ref\|WP_003806748.1\| | ligase [Bifidobacterium | 97.692 | 260 |
| | | adolescentis] | | |
| Z25_GM001716 | gi\|500063597\|ref\|WP_011743514.1\| | LacI family transcriptional | 99.712 | 347 |
| | | regulator [Bifidobacterium | | |
| | | adolescentis] | | |
| Z25_GM001722 | gi\|705408847\|ref\|WP_033499910.1\| | deoxyribonuclease HsdR | 98.844 | 1038 |
| | | [Bifidobacterium adolescentis] | | |
| Z25_GM001732 | gi\|751369233\|gb\|KIM01747.1\| | alpha/beta hydrolase [Bifidobacterium | 97.902 | 286 |
| | | adolescentis] | | |
| Z25_GM001737 | gi\|748204973\|ref\|WP_039775661.1\| | hypothetical protein [Bifidobacterium | 99.643 | 561 |
| | | adolescentis] | | |
| Z25_GM001738 | gi\|822581535\|gb\|KLE27047.1\| | exopolysaccharide biosynthesis | 100 | 554 |
| | | polyprenyl glycosylphosphotransferase | | |
| | | [Bifidobacterium adolescentis] | | |
| Z25_GM001741 | gi\|671343263\|gb\|AII76779.1\| | hypothetical protein BADO_1371 | 97.692 | 130 |
| | | [Bifidobacterium adolescentis] | | |
| Z25_GM001742 | gi\|822625628\|ref\|WP_046999683.1\| | hypothetical protein [Bifidobacterium | 100 | 61 |
| | | adolescentis] | | |
| Z25_GM001744 | gi\|822625629\|ref\|WP_046999684.1\| | hypothetical protein [Bifidobacterium | 100 | 191 |
| | | adolescentis] | | |
| Z25_GM001745 | gi\|920095976\|ref\|WP_052946241.1\| | hypothetical protein [Bifidobacterium | 99.296 | 142 |
| | | adolescentis] | | |
| Z25_GM001761 | gi\|695759186\|ref\|WP_032682397.1\| | nitroreductase [Bifidobacterium | 44.326 | 282 |
| | | longum] | | |
| Z25_GM001762 | gi\|651887594\|ref\|WP_026647221.1\| | MULTISPECIES: multidrug | 98.96 | 481 |
| | | transporter [Bifidobacterium] | | |
| Z25_GM001763 | gi\|547053514\|ref\|WP_021912793.1\| | MULTISPECIES: hypothetical | 99.257 | 269 |
| | | protein [Bifidobacterium] | | |
| Z25_GM001765 | gi\|822625640\|ref\|WP_046999695.1\| | integrase [Bifidobacterium | 100 | 31 |
| | | adolescentis] | | |
| Z25_GM001771 | gi\|748204999\|ref\|WP_039775687.1\| | hypothetical protein [Bifidobacterium | 97.727 | 264 |
| | | adolescentis] | | |
| Z25_GM001774 | gi\|748205357\|ref\|WP_039776045.1\| | MFS transporter [Bifidobacterium | 100 | 394 |
| | | adolescentis] | | |
| Z25_GM001776 | gi\|917738583\|ref\|WP_052252769.1\| | hypothetical protein [Bifidobacterium | 100 | 69 |
| | | adolescentis] | | |
| Z25_GM001782 | gi\|489906168\|ref\|WP_003809592.1\| | hemolysin III [Bifidobacterium | 100 | 295 |
| | | adolescentis] | | |
| Z25_GM001787 | gi\|489905617\|ref\|WP_003809042.1\| | hypothetical protein [Bifidobacterium | 75.972 | 283 |
| | | adolescentis] | | |
| Z25_GM001790 | gi\|673000931\|gb\|KFI95998.1\| | hypothetical protein BSTER_1710 | 90.566 | 53 |
| | | [Bifidobacterium stercoris | | |
| | | JCM 15918] | | |
| Z25_GM001794 | gi\|747123691\|gb\|AJE05113.1\| | Mobile element protein [Bifidobacterium | 99.664 | 298 |
| | | adolescentis] | | |

| | | | | | Initial | End | | |
|---|---|---|---|---|---|---|---|---|
| | | | Initial | End | Database | Database | | |
| Gene ID | Mismatching | Gap | Gene | Gene | Article | Article | E | Score |
| Z25_GM000009 | 29 | 0 | 37 | 405 | 19 | 141 | 2.65E−66 | 196 |
| Z25_GM000013 | 16 | 0 | 1 | 384 | 1 | 128 | 1.10E−70 | 206 |
| Z25_GM000014 | 4 | 0 | 1 | 243 | 1 | 81 | 2.17E−51 | 154 |
| Z25_GM000019 | 0 | 0 | 1 | 243 | 1 | 81 | 6.14E−58 | 170 |
| Z25_GM000024 | 32 | 0 | 1 | 531 | 1 | 177 | 4.98E−79 | 231 |
| Z25_GM000026 | 7 | 0 | 1 | 189 | 1 | 63 | 2.54E−36 | 114 |
| Z25_GM000029 | 48 | 2 | 49 | 285 | 6 | 82 | 5.56E−09 | 47.4 |
| Z25_GM000031 | 26 | 1 | 1 | 162 | 1 | 55 | 2.22E−11 | 51.2 |
| Z25_GM000032 | 20 | 0 | 1 | 132 | 1 | 44 | 1.57E−13 | 56.6 |
| Z25_GM000034 | 24 | 0 | 154 | 858 | 1 | 235 | 1.33E−157 | 437 |
| Z25_GM000035 | 6 | 0 | 1 | 327 | 1 | 109 | 4.39E−67 | 196 |
| Z25_GM000038 | 287 | 11 | 97 | 1464 | 16 | 488 | 7.28E−77 | 249 |
| Z25_GM000039 | 116 | 5 | 70 | 738 | 24 | 241 | 5.77E−59 | 199 |
| Z25_GM000047 | 0 | 0 | 1 | 618 | 1 | 206 | 2.58E−153 | 421 |
| Z25_GM000048 | 3 | 0 | 1 | 378 | 1 | 126 | 1.77E−90 | 256 |
| Z25_GM000049 | 0 | 0 | 1 | 198 | 1 | 66 | 4.55E−46 | 139 |
| Z25_GM000053 | 9 | 1 | 1 | 408 | 1 | 139 | 4.92E−65 | 202 |
| Z25_GM000053 | 8 | 0 | 676 | 837 | 202 | 255 | 1.28E−23 | 95.1 |
| Z25_GM000054 | 62 | 5 | 1 | 549 | 1 | 188 | 2.63E−29 | 105 |
| Z25_GM000058 | 5 | 0 | 25 | 288 | 1 | 88 | 1.82E−57 | 170 |
| Z25_GM000059 | 2 | 0 | 154 | 954 | 1 | 267 | 0 | 538 |
| Z25_GM000079 | 0 | 0 | 1 | 234 | 1 | 78 | 1.70E−47 | 144 |
| Z25_GM000121 | 0 | 0 | 1 | 2304 | 60 | 827 | 0 | 1498 |
| Z25_GM000122 | 0 | 0 | 1 | 411 | 1 | 137 | 2.21E−102 | 287 |
| Z25_GM000123 | 1 | 0 | 1 | 999 | 1 | 333 | 0 | 627 |
| Z25_GM000155 | 0 | 0 | 133 | 507 | 1 | 125 | 2.93E−88 | 252 |
| Z25_GM000158 | 0 | 0 | 1 | 1506 | 1 | 502 | 0 | 979 |
| Z25_GM000206 | 0 | 0 | 1 | 594 | 9 | 206 | 3.90E−149 | 410 |
| Z25_GM000208 | 0 | 0 | 1 | 147 | 15 | 63 | 2.87E−32 | 103 |
| Z25_GM000214 | 0 | 0 | 1 | 1143 | 1 | 381 | 0 | 713 |
| Z25_GM000236 | 0 | 0 | 158 | 51 | 8 | 43 | 1.89E−20 | 74.3 |
| Z25_GM000237 | 0 | 0 | 1 | 252 | 1 | 84 | 1.96E−57 | 169 |
| Z25_GM000262 | 1 | 0 | 1 | 537 | 1 | 179 | 1.51E−132 | 367 |
| Z25_GM000266 | 0 | 0 | 114 | 1 | 1 | 38 | 4.15E−21 | 76.3 |
| Z25_GM000267 | 0 | 0 | 1 | 213 | 1 | 71 | 9.96E−50 | 149 |
| Z25_GM000273 | 0 | 0 | 1 | 336 | 1 | 112 | 8.14E−79 | 225 |
| Z25_GM000279 | 0 | 0 | 1 | 276 | 1 | 92 | 7.79E−66 | 191 |
| Z25_GM000289 | 0 | 0 | 1 | 882 | 1 | 294 | 0 | 613 |
| Z25_GM000297 | 0 | 0 | 1 | 2730 | 1 | 910 | 0 | 1768 |
| Z25_GM000298 | 0 | 0 | 1 | 987 | 1 | 329 | 0 | 677 |
| Z25_GM000300 | 0 | 0 | 1 | 702 | 272 | 505 | 1.69E−176 | 493 |
| Z25_GM000302 | 1 | 0 | 1 | 2022 | 1 | 674 | 0 | 1389 |
| Z25_GM000303 | 1 | 0 | 1 | 456 | 1 | 152 | 5.39E−111 | 310 |
| Z25_GM000304 | 0 | 0 | 31 | 129 | 4 | 36 | 1.26E−19 | 70.9 |
| Z25_GM000306 | 0 | 0 | 1 | 126 | 1 | 42 | 6.35E−25 | 84.3 |
| Z25_GM000308 | 8 | 0 | 1 | 129 | 1 | 43 | 2.79E−20 | 72.8 |
| Z25_GM000313 | 4 | 0 | 1 | 942 | 1 | 314 | 0 | 633 |
| Z25_GM000321 | 0 | 0 | 58 | 786 | 18 | 260 | 1.58E−160 | 444 |
| Z25_GM000322 | 1 | 0 | 1 | 507 | 1 | 169 | 1.01E−126 | 351 |
| Z25_GM000325 | 0 | 0 | 1 | 516 | 1 | 172 | 2.34E−123 | 343 |
| Z25_GM000331 | 6 | 0 | 1 | 681 | 1 | 227 | 9.73E−147 | 408 |
| Z25_GM000332 | 3 | 0 | 1 | 2601 | 1 | 867 | 0 | 1765 |
| Z25_GM000341 | 0 | 0 | 1 | 786 | 1 | 262 | 1.94E−167 | 462 |
| Z25_GM000345 | 0 | 0 | 1 | 876 | 1 | 292 | 0 | 590 |
| Z25_GM000356 | 0 | 0 | 1 | 1080 | 1 | 360 | 0 | 743 |
| Z25_GM000361 | 5 | 0 | 1 | 3783 | 1 | 1261 | 0 | 2506 |
| Z25_GM000364 | 0 | 0 | 1 | 723 | 12 | 252 | 2.05E−161 | 445 |
| Z25_GM000373 | 0 | 0 | 1 | 744 | 1 | 248 | 4.34E−167 | 460 |
| Z25_GM000375 | 0 | 0 | 1 | 183 | 25 | 85 | 3.63E−41 | 127 |
| Z25_GM000380 | 1 | 0 | 1 | 621 | 1 | 207 | 2.78E−153 | 421 |
| Z25_GM000381 | 0 | 0 | 1 | 882 | 23 | 316 | 0 | 615 |
| Z25_GM000383 | 0 | 0 | 1 | 327 | 1 | 109 | 3.25E−77 | 221 |
| Z25_GM000390 | 1 | 0 | 1 | 207 | 29 | 97 | 2.31E−47 | 144 |
| Z25_GM000401 | 0 | 0 | 1 | 309 | 1 | 103 | 4.96E−74 | 213 |
| Z25_GM000415 | 0 | 0 | 1 | 129 | 1 | 43 | 4.04E−27 | 90.1 |
| Z25_GM000420 | 0 | 0 | 1 | 693 | 1 | 231 | 5.65E−154 | 425 |
| Z25_GM000421 | 0 | 0 | 49 | 1059 | 17 | 353 | 0 | 684 |
| Z25_GM000423 | 0 | 0 | 1 | 507 | 1 | 169 | 1.18E−102 | 290 |
| Z25_GM000432 | 0 | 0 | 1 | 972 | 1 | 324 | 0 | 588 |
| Z25_GM000464 | 0 | 0 | 1 | 216 | 14 | 85 | 7.51E−49 | 147 |
| Z25_GM000470 | 0 | 0 | 1 | 801 | 3 | 269 | 0 | 521 |
| Z25_GM000472 | 0 | 0 | 1 | 951 | 1 | 317 | 0 | 654 |
| Z25_GM000474 | 0 | 0 | 1 | 192 | 1 | 64 | 1.06E−40 | 125 |
| Z25_GM000482 | 0 | 0 | 1 | 237 | 5 | 83 | 7.26E−38 | 120 |
| Z25_GM000496 | 2 | 0 | 53 | 193 | 1 | 47 | 9.18E−29 | 96.7 |
| Z25_GM000506 | 1 | 0 | 1 | 1800 | 1 | 600 | 0 | 1159 |
| Z25_GM000509 | 0 | 0 | 1 | 513 | 1 | 171 | 4.07E−126 | 350 |
| Z25_GM000510 | 1 | 0 | 1 | 195 | 1 | 65 | 2.19E−44 | 135 |
| Z25_GM000513 | 0 | 0 | 1 | 450 | 21 | 170 | 2.59E−111 | 311 |
| Z25_GM000514 | 0 | 0 | 1 | 255 | 17 | 101 | 4.79E−60 | 176 |
| Z25_GM000561 | 0 | 0 | 1 | 789 | 1 | 263 | 0 | 514 |
| Z25_GM000577 | 12 | 0 | 1 | 834 | 1 | 278 | 0 | 541 |
| Z25_GM000579 | 0 | 0 | 1 | 177 | 17 | 75 | 7.99E−39 | 121 |
| Z25_GM000580 | 3 | 0 | 1 | 180 | 72 | 131 | 3.69E−38 | 121 |
| Z25_GM000581 | 0 | 0 | 1 | 222 | 26 | 99 | 6.30E−50 | 151 |
| Z25_GM000582 | 0 | 0 | 40 | 825 | 14 | 275 | 0 | 546 |
| Z25_GM000583 | 0 | 0 | 1 | 144 | 1 | 48 | 1.13E−30 | 99.4 |
| Z25_GM000590 | 0 | 0 | 1 | 192 | 1 | 64 | 7.89E−44 | 133 |
| Z25_GM000655 | 2 | 0 | 1 | 270 | 1 | 90 | 1.83E−62 | 182 |
| Z25_GM000657 | 1 | 0 | 1 | 180 | 1 | 60 | 9.01E−41 | 125 |
| Z25_GM000667 | 4 | 0 | 1 | 357 | 56 | 174 | 4.09E−82 | 236 |
| Z25_GM000668 | 210 | 24 | 172 | 1380 | 98 | 465 | 7.16E−26 | 110 |
| Z25_GM000669 | 5 | 0 | 1 | 990 | 1 | 330 | 0 | 593 |
| Z25_GM000670 | 0 | 0 | 1 | 573 | 66 | 256 | 4.03E−134 | 375 |
| Z25_GM000671 | 64 | 0 | 10 | 846 | 1 | 279 | 2.38E−165 | 459 |
| Z25_GM000752 | 2 | 0 | 1 | 1878 | 1 | 626 | 0 | 1306 |
| Z25_GM000764 | 4 | 0 | 1 | 249 | 10 | 92 | 1.03E−53 | 160 |
| Z25_GM000770 | 8 | 0 | 1 | 366 | 1 | 122 | 3.64E−82 | 234 |
| Z25_GM000771 | 10 | 1 | 1 | 408 | 1 | 137 | 1.80E−90 | 258 |
| Z25_GM000772 | 5 | 0 | 1 | 168 | 1 | 56 | 2.67E−31 | 101 |
| Z25_GM000774 | 34 | 1 | 1 | 579 | 1 | 192 | 3.83E−115 | 324 |
| Z25_GM000776 | 92 | 2 | 16 | 903 | 5 | 301 | 9.22E−146 | 410 |
| Z25_GM000780 | 110 | 4 | 1 | 588 | 1 | 199 | 3.32E−48 | 155 |
| Z25_GM000782 | 27 | 0 | 13 | 279 | 6 | 94 | 7.78E−41 | 128 |
| Z25_GM000783 | 53 | 2 | 1 | 390 | 1 | 132 | 1.84E−41 | 132 |
| Z25_GM000784 | 1 | 0 | 1 | 513 | 1 | 171 | 5.25E−125 | 347 |
| Z25_GM000789 | 0 | 0 | 1 | 1611 | 1 | 537 | 0 | 1111 |
| Z25_GM000791 | 7 | 0 | 1 | 720 | 11 | 250 | 7.22E−175 | 479 |
| Z25_GM000793 | 5 | 0 | 1 | 156 | 1 | 52 | 9.01E−33 | 105 |
| Z25_GM000795 | 4 | 0 | 4 | 174 | 1 | 57 | 3.49E−35 | 111 |
| Z25_GM000796 | 4 | 0 | 1 | 132 | 1 | 44 | 1.78E−26 | 88.6 |
| Z25_GM000798 | 2 | 0 | 1 | 252 | 1 | 84 | 3.16E−58 | 171 |
| Z25_GM000799 | 6 | 0 | 1 | 204 | 1 | 68 | 9.58E−43 | 131 |
| Z25_GM000800 | 0 | 0 | 490 | 972 | 1 | 161 | 6.20E−122 | 345 |
| Z25_GM000801 | 0 | 0 | 1 | 375 | 1 | 125 | 1.91E−91 | 259 |
| Z25_GM000802 | 1 | 0 | 1 | 162 | 1 | 54 | 3.24E−34 | 108 |
| Z25_GM000808 | 10 | 0 | 1 | 177 | 1 | 59 | 1.71E−31 | 102 |
| Z25_GM000809 | 6 | 0 | 10 | 219 | 3 | 72 | 1.09E−44 | 136 |
| Z25_GM000810 | 87 | 6 | 1 | 762 | 1 | 261 | 4.83E−108 | 311 |
| Z25_GM000811 | 31 | 1 | 1 | 600 | 1 | 185 | 2.83E−109 | 309 |
| Z25_GM000815 | 2 | 0 | 1 | 225 | 1 | 75 | 1.17E−50 | 151 |
| Z25_GM000817 | 4 | 0 | 1 | 126 | 1 | 42 | 3.67E−25 | 85.1 |
| Z25_GM000818 | 8 | 0 | 1 | 360 | 1 | 120 | 3.53E−83 | 237 |
| Z25_GM000822 | 103 | 4 | 10 | 480 | 3 | 162 | 2.24E−10 | 54.7 |
| Z25_GM000834 | 0 | 0 | 1 | 84 | 53 | 80 | 2.75E−16 | 63.2 |
| Z25_GM000835 | 0 | 0 | 1 | 513 | 1 | 171 | 2.17E−128 | 355 |
| Z25_GM000856 | 97 | 11 | 1 | 609 | 4 | 197 | 7.45E−31 | 115 |
| Z25_GM000856 | 108 | 8 | 16 | 582 | 204 | 382 | 1.92E−16 | 75.5 |
| Z25_GM000858 | 1 | 0 | 1 | 924 | 1 | 308 | 0 | 639 |
| Z25_GM000870 | 4 | 0 | 160 | 465 | 1 | 102 | 1.86E−57 | 174 |
| Z25_GM000873 | 25 | 0 | 4 | 318 | 28 | 132 | 4.52E−57 | 171 |
| Z25_GM000874 | 6 | 0 | 1084 | 1272 | 1 | 63 | 1.81E−18 | 78.6 |
| Z25_GM000881 | 12 | 0 | 1 | 2607 | 1 | 869 | 0 | 1582 |
| Z25_GM000915 | 0 | 0 | 1 | 1005 | 20 | 354 | 0 | 683 |
| Z25_GM000921 | 0 | 0 | 1 | 288 | 1 | 96 | 4.31E−64 | 187 |
| Z25_GM000926 | 0 | 0 | 1 | 951 | 1 | 317 | 0 | 647 |
| Z25_GM000927 | 0 | 0 | 1 | 522 | 1 | 174 | 6.61E−106 | 299 |
| Z25_GM000928 | 1 | 0 | 1 | 1065 | 1 | 355 | 0 | 638 |
| Z25_GM000930 | 1 | 0 | 1 | 555 | 6 | 190 | 4.96E−114 | 320 |
| Z25_GM000932 | 4 | 0 | 1 | 1557 | 1 | 519 | 0 | 1074 |
| Z25_GM000943 | 0 | 0 | 1 | 681 | 1 | 227 | 9.13E−167 | 457 |
| Z25_GM000966 | 0 | 0 | 1 | 225 | 1 | 75 | 4.37E−52 | 155 |
| Z25_GM001016 | 72 | 8 | 1 | 1575 | 1 | 526 | 0 | 711 |
| Z25_GM001017 | 0 | 0 | 16 | 567 | 1 | 184 | 1.11E−112 | 317 |
| Z25_GM001020 | 14 | 0 | 1 | 2184 | 157 | 884 | 0 | 1417 |
| Z25_GM001042 | 0 | 0 | 1 | 675 | 1 | 225 | 3.63E−167 | 458 |
| Z25_GM001043 | 1 | 0 | 1 | 2193 | 1 | 731 | 0 | 1197 |
| Z25_GM001046 | 0 | 0 | 1 | 234 | 1 | 78 | 3.93E−55 | 163 |
| Z25_GM001051 | 1 | 0 | 1 | 171 | 1 | 57 | 2.46E−25 | 86.7 |
| Z25_GM001067 | 0 | 0 | 1 | 804 | 5 | 272 | 1.01E−178 | 491 |
| Z25_GM001068 | 5 | 0 | 1 | 1887 | 29 | 657 | 0 | 1269 |
| Z25_GM001069 | 24 | 0 | 1 | 4698 | 14 | 1579 | 0 | 3036 |
| Z25_GM001073 | 2 | 0 | 1 | 774 | 1 | 258 | 1.07E−156 | 434 |
| Z25_GM001149 | 0 | 0 | 37 | 405 | 1 | 123 | 1.23E−66 | 196 |
| Z25_GM001157 | 0 | 0 | 1 | 816 | 1 | 272 | 0 | 561 |
| Z25_GM001178 | 0 | 0 | 1 | 2088 | 1 | 696 | 0 | 1439 |
| Z25_GM001194 | 1 | 0 | 131 | 3 | 1 | 43 | 1.67E−25 | 87 |
| Z25_GM001220 | 6 | 0 | 1 | 1605 | 1 | 535 | 0 | 1061 |
| Z25_GM001244 | 0 | 0 | 1 | 996 | 1 | 332 | 0 | 648 |
| Z25_GM001245 | 0 | 0 | 1 | 1089 | 1 | 363 | 0 | 604 |
| Z25_GM001248 | 0 | 0 | 1 | 276 | 1 | 92 | 1.44E−63 | 185 |
| Z25_GM001252 | 0 | 0 | 1 | 825 | 1 | 275 | 0 | 565 |
| Z25_GM001266 | 0 | 0 | 1 | 1929 | 15 | 657 | 0 | 1064 |
| Z25_GM001268 | 0 | 0 | 1 | 474 | 1 | 158 | 6.53E−115 | 320 |
| Z25_GM001270 | 3 | 0 | 64 | 342 | 1 | 93 | 6.71E−55 | 164 |
| Z25_GM001276 | 0 | 0 | 1 | 294 | 1 | 98 | 4.48E−67 | 195 |
| Z25_GM001282 | 1 | 0 | 1 | 819 | 1 | 273 | 0 | 559 |
| Z25_GM001286 | 0 | 0 | 1 | 816 | 1 | 272 | 1.57E−178 | 491 |
| Z25_GM001301 | 40 | 1 | 220 | 420 | 19 | 84 | 3.90E−10 | 53.5 |
| Z25_GM001321 | 0 | 0 | 1 | 741 | 1 | 247 | 0 | 495 |
| Z25_GM001341 | 0 | 0 | 1 | 465 | 1 | 155 | 8.77E−100 | 282 |
| Z25_GM001366 | 0 | 0 | 1 | 147 | 1 | 49 | 6.56E−31 | 100 |
| Z25_GM001377 | 1 | 0 | 1 | 1053 | 1 | 351 | 0 | 659 |
| Z25_GM001381 | 10 | 0 | 1 | 2322 | 3 | 776 | 0 | 1405 |
| Z25_GM001382 | 10 | 0 | 1 | 2709 | 1 | 903 | 0 | 1678 |
| Z25_GM001387 | 0 | 0 | 1 | 126 | 1 | 42 | 2.52E−25 | 85.5 |
| Z25_GM001396 | 0 | 0 | 1 | 804 | 33 | 300 | 0 | 507 |
| Z25_GM001397 | 0 | 0 | 1 | 222 | 1 | 74 | 1.52E−49 | 149 |
| Z25_GM001403 | 8 | 0 | 1 | 1572 | 1 | 524 | 0 | 710 |
| Z25_GM001406 | 0 | 0 | 1 | 1623 | 1 | 541 | 0 | 1044 |
| Z25_GM001421 | 1 | 0 | 1 | 270 | 1 | 90 | 2.11E−62 | 182 |
| Z25_GM001422 | 1 | 0 | 1 | 624 | 1 | 208 | 1.54E−138 | 384 |
| Z25_GM001426 | 2 | 0 | 1 | 1803 | 1 | 601 | 0 | 1074 |
| Z25_GM001427 | 0 | 0 | 1 | 1773 | 1 | 591 | 0 | 1187 |
| Z25_GM001428 | 0 | 0 | 1 | 792 | 1 | 264 | 0 | 543 |
| Z25_GM001429 | 0 | 0 | 1 | 573 | 1 | 191 | 6.57E−140 | 386 |
| Z25_GM001432 | 0 | 0 | 1 | 2415 | 1 | 805 | 0 | 1633 |
| Z25_GM001436 | 0 | 0 | 1 | 609 | 1 | 203 | 1.76E−139 | 386 |
| Z25_GM001441 | 1 | 0 | 1 | 219 | 1 | 73 | 2.47E−46 | 142 |
| Z25_GM001446 | 0 | 0 | 8 | 88 | 1 | 27 | 7.28E−15 | 63.5 |
| Z25_GM001449 | 0 | 0 | 1 | 597 | 1 | 199 | 6.41E−147 | 405 |
| Z25_GM001456 | 2 | 0 | 1 | 543 | 38 | 218 | 4.72E−137 | 380 |
| Z25_GM001457 | 33 | 0 | 7 | 651 | 4 | 218 | 8.47E−135 | 375 |
| Z25_GM001458 | 9 | 0 | 922 | 1191 | 10 | 99 | 2.90E−55 | 176 |
| Z25_GM001460 | 15 | 0 | 70 | 624 | 52 | 236 | 7.40E−125 | 351 |
| Z25_GM001467 | 0 | 0 | 1 | 249 | 1 | 83 | 4.13E−61 | 178 |
| Z25_GM001481 | 0 | 0 | 1 | 897 | 1 | 299 | 0 | 576 |
| Z25_GM001483 | 1 | 0 | 1 | 747 | 1 | 249 | 8.18E−170 | 467 |
| Z25_GM001489 | 5 | 0 | 109 | 1035 | 49 | 357 | 0 | 543 |
| Z25_GM001496 | 0 | 0 | 1 | 534 | 25 | 202 | 2.64E−98 | 281 |
| Z25_GM001497 | 0 | 0 | 1 | 165 | 20 | 74 | 4.83E−37 | 117 |
| Z25_GM001499 | 1 | 0 | 1 | 1239 | 1 | 413 | 0 | 848 |
| Z25_GM001508 | 0 | 0 | 1 | 2412 | 1 | 804 | 0 | 1629 |
| Z25_GM001520 | 1 | 0 | 1 | 102 | 1 | 34 | 7.55E−19 | 68.6 |
| Z25_GM001532 | 0 | 0 | 207 | 1 | 14 | 82 | 5.66E−46 | 145 |
| Z25_GM001533 | 4 | 0 | 1 | 2847 | 126 | 1074 | 0 | 1842 |
| Z25_GM001536 | 0 | 0 | 1 | 447 | 1 | 149 | 7.52E−93 | 264 |
| Z25_GM001540 | 316 | 11 | 4 | 1311 | 47 | 477 | 4.60E−15 | 75.5 |
| Z25_GM001557 | 0 | 0 | 1 | 711 | 1 | 237 | 0 | 495 |
| Z25_GM001559 | 0 | 0 | 1 | 1269 | 1 | 423 | 0 | 876 |
| Z25_GM001567 | 95 | 6 | 127 | 603 | 379 | 537 | 6.65E−07 | 51.2 |
| Z25_GM001569 | 0 | 0 | 81 | 1 | 5 | 31 | 7.82E−15 | 58.9 |
| Z25_GM001573 | 0 | 0 | 1 | 633 | 1 | 211 | 1.15E−159 | 438 |
| Z25_GM001581 | 1 | 0 | 1 | 684 | 1 | 228 | 1.18E−154 | 427 |
| Z25_GM001595 | 1 | 0 | 1 | 615 | 1 | 205 | 1.38E−154 | 424 |
| Z25_GM001609 | 1 | 0 | 22 | 135 | 1 | 38 | 1.15E−22 | 79 |
| Z25_GM001611 | 1 | 0 | 1 | 927 | 1 | 309 | 0 | 637 |
| Z25_GM001612 | 3 | 0 | 1 | 579 | 201 | 393 | 6.42E−132 | 374 |
| Z25_GM001613 | 9 | 0 | 1 | 969 | 1 | 323 | 0 | 649 |
| Z25_GM001647 | 1 | 0 | 1 | 528 | 1 | 176 | 8.95E−131 | 362 |
| Z25_GM001648 | 3 | 0 | 1 | 822 | 1 | 274 | 6.97E−157 | 436 |
| Z25_GM001649 | 3 | 0 | 1 | 1971 | 19 | 675 | 0 | 1338 |
| Z25_GM001651 | 82 | 1 | 1 | 1266 | 1 | 418 | 0 | 697 |
| Z25_GM001652 | 33 | 0 | 25 | 858 | 1 | 278 | 5.94E−165 | 457 |
| Z25_GM001653 | 66 | 0 | 1 | 1857 | 4 | 622 | 0 | 1181 |
| Z25_GM001655 | 24 | 0 | 3 | 185 | 159 | 219 | 4.31E−20 | 78.2 |
| Z25_GM001658 | 209 | 10 | 4 | 1107 | 42 | 389 | 1.42E−53 | 189 |
| Z25_GM001660 | 341 | 18 | 4 | 1764 | 52 | 621 | 1.36E−79 | 261 |
| Z25_GM001662 | 88 | 5 | 420 | 1 | 151 | 301 | 6.23E−17 | 75.5 |
| Z25_GM001664 | 35 | 0 | 1 | 939 | 2 | 314 | 0 | 552 |
| Z25_GM001665 | 236 | 10 | 1 | 1062 | 157 | 509 | 1.95E−28 | 115 |
| Z25_GM001677 | 304 | 2 | 1 | 2127 | 1 | 705 | 0 | 829 |
| Z25_GM001678 | 187 | 3 | 82 | 942 | 30 | 315 | 2.44E−44 | 152 |
| Z25_GM001686 | 0 | 0 | 1 | 840 | 1 | 280 | 2.97E−164 | 455 |
| Z25_GM001691 | 0 | 0 | 1 | 990 | 29 | 358 | 0 | 628 |
| Z25_GM001694 | 0 | 0 | 1 | 318 | 1 | 106 | 1.83E−74 | 214 |
| Z25_GM001698 | 1 | 0 | 1 | 1458 | 2 | 487 | 0 | 891 |
| Z25_GM001702 | 0 | 0 | 1 | 213 | 1 | 71 | 4.51E−50 | 151 |
| Z25_GM001710 | 6 | 0 | 1 | 780 | 186 | 445 | 0 | 529 |
| Z25_GM001716 | 1 | 0 | 1 | 1041 | 1 | 347 | 0 | 711 |
| Z25_GM001722 | 12 | 0 | 1 | 3114 | 1 | 1038 | 0 | 2115 |
| Z25_GM001732 | 2 | 1 | 1 | 846 | 1 | 286 | 0 | 566 |
| Z25_GM001737 | 2 | 0 | 55 | 1737 | 35 | 595 | 0 | 1087 |
| Z25_GM001738 | 0 | 0 | 1 | 1662 | 1 | 554 | 0 | 1074 |
| Z25_GM001741 | 3 | 0 | 1 | 390 | 1 | 130 | 1.96E−94 | 266 |
| Z25_GM001742 | 0 | 0 | 185 | 3 | 1 | 61 | 7.52E−28 | 101 |
| Z25_GM001744 | 0 | 0 | 1 | 573 | 28 | 218 | 1.22E−133 | 372 |
| Z25_GM001745 | 1 | 0 | 1 | 426 | 18 | 159 | 3.99E−90 | 257 |
| Z25_GM001761 | 157 | 0 | 1 | 846 | 52 | 333 | 1.64E−86 | 260 |
| Z25_GM001762 | 5 | 0 | 1 | 1443 | 1 | 481 | 0 | 825 |
| Z25_GM001763 | 2 | 0 | 1 | 807 | 103 | 371 | 0 | 554 |
| Z25_GM001765 | 0 | 0 | 1 | 93 | 28 | 58 | 3.38E−18 | 68.2 |
| Z25_GM001771 | 6 | 0 | 4 | 795 | 1 | 264 | 0 | 499 |
| Z25_GM001774 | 0 | 0 | 1 | 1182 | 18 | 411 | 0 | 761 |
| Z25_GM001776 | 0 | 0 | 1 | 207 | 1 | 69 | 6.78E−48 | 144 |
| Z25_GM001782 | 0 | 0 | 1 | 885 | 1 | 295 | 0 | 529 |
| Z25_GM001787 | 68 | 0 | 1 | 849 | 1 | 283 | 3.32E−161 | 448 |
| Z25_GM001790 | 5 | 0 | 49 | 207 | 1 | 53 | 2.69E−32 | 104 |
| Z25_GM001794 | 1 | 0 | 1 | 894 | 1 | 298 | 0 | 584 |

The beneficial technical effects of the present disclosure are as follows.

The strain CCFM8630 of Bifidobacterium adolescentis of the present disclosure significantly increases neurotransmitter 5-hydroxytryptamine (5-HT) level in peripheral blood of rat, regulates brain-gut axis, relieves mental illnesses related to metabolic syndrome, for example anxiety, depression and so on, recovers the hormone level, for example testosterone and so on in peripheral blood of rat caused by high-fat high-starch diet, recovers abundances of Bifidobacterium genus, Blautia genus and Turicibacter genus in abnormal intestinal flora of rat caused by high-fat high-starch diet. In addition, strain CCFM8630 of Bifidobacterium adolescentis has pretty good tolerance to simulated gastrointestinal fluid, and quickly colonizes in intestinal, significantly alleviates pathology damages of tissues, such as liver, duodenum and so on of rat with metabolic syndrome caused by high-fat high-starch diet; significantly improves oral glucose tolerance of rat with metabolic syndrome and decreases the under curve area of glucose tolerance test; significantly increases triglyceride and total cholesterol levels in serum of rat with metabolic syndrome caused by high-fat high-starch diet. The strain CCFM8630 of Bifidobacterium adolescentis of the present disclosure can be used to prepare health foods or medicines that improve metabolic syndrome, regulates intestinal flora, relieves irritable bowel syndrome, regulates brain-gut axis and alleviates mental illness such as anxiety, depression and so on, which has a pretty wide application prospect.

In order to understand the present disclosure further, the technical solutions in the examples of the present disclosure will be described clearly and completely herein in conjunction with the examples of the present disclosure. Apparently, the described examples are only a part of the examples of the present disclosure, rather than all examples. Based on the examples in the present disclosure, all of other examples, made by one of ordinary skill in the art without any creative efforts, fall into the protection scope of the present disclosure.

All of the reagents related to examples of the present disclosure are commercial products without special description, which can be purchased on market. All of the following examples are completed by theory and technology research group of probiotics of Research Center of Food Biotechnology in School of Food Science and Technology, Jiangnan University.

### Example 1: Strain CCFM8630 of Bifidobacterium adolescentis has Good Tolerance to Simulated Gastrointestinal Fluid

The cryopreserved strain CCFM8630 of Bifidobacterium adolescentis were inoculated in the mMRS medium (MRS medium containing 0.05% cysteine hydrochloride) and cultured at 37° C. for 48 hours under anaerobic cultivation, followed by 2 to 3 times subculture in mMRS liquid medium. The medium with strain CCFM8630 of Bifidobacterium adolescentis was taken and centrifuged for 5 minutes at a speed of 8000×g, and then resuspended (1:1) in an artificial simulated gastric juice (mMRS medium containing 1% pepsin, pH 2.5), followed by anaerobic cultivation at 37° C. Sampling was carried out at 0 hour, 0.5 hour, 1 hour and 2 hours, and the samples were cultured on mMRS medium agar plate for colony counting. The viability numbers were counted and the survival rates were calculated. The survival rate is the rate of the viable count at the desired time point to the viable count at the 0 hour, which was expressed in %.

The medium with cultured strain CCFM8630 of Bifidobacterium adolescentis was taken and centrifuged at a speed of 8000×g for 5 minutes. The bacteria were collected and resuspened (1:1) in artificial simulated intestinal fluid (mMRS medium containing 0.3% bile salt from ox, 1% trypsin, pH 8.0), followed by anaerobic cultivation at 37° C. Sampling was carried out at 0 hour, 0.5 hour, 1 hour, 2 hours, 3 hours and 4 hours, and the samples were cultured on mMRS medium agar plate for colony counting. The viability numbers were counted and the survival rates were calculated. The survival rate is the rate of the viable count at the desired time point to the viable count at the 0 hour, which was expressed in %.

The experiment results were shown in Table 2 and Table 3. The results showed that strain CCFM8630 of Bifidobacterium adolescentis has a relative good tolerance to simulated gastrointestinal fluid.

TABLE 2
Tolerance of strain CCFM8630 of Bifidobacterium adolescentis to
simulated gastrointestinal fluid

| | Simulated Gastric Fluid | | |
|---|---|---|---|
| | Treatment Time (h) | | |
| | 0.5 | 1 | 2 |
| Survival Rate (%) | 56.9 | 39.5 | 12.6 |

TABLE 3
Tolerance of strain CCFM8630 of Bifidobacterium adolescentis to
simulated intestinal fluid

| | Simulated Intestinal Fluid | | | | |
|---|---|---|---|---|---|
| | Treatment Time (h) | | | | |
| | 0.5 | 1 | 2 | 3 | 4 |
| Survival Rate (%) | 100 | 100 | 49.1 | 51.6 | 25.9 |

### Example 2: Strain CCFM8630 of Bifidobacterium adolescentis has No Toxic and Side Effects on SD Rat

The strain CCFM8630 of Bifidobacterium adolescentis bacteria were resuspended in 2% sucrose solution to give a bacterial suspension with a concentration of 3.0×10⁹ CFU/mL. 8 healthy male SD rats with a weight between 180 and 200 g were chosen and acclimated for 1 week before experiments. The rats were administered with the above bacteria suspension by intragastric gavage once daily at a dose of 2 mL/day/rat. The death and weight of the rats were observed and recorded for one week. The results were shown in Table 4.

TABLE 4
Death and changes of body weight in rats

| Time (day) | 1 | 2 | 3 | 4 | 5 | 6 | 7 |
|---|---|---|---|---|---|---|---|
| Weight (g) | 230.2 ± 1.2 | 234.8±1.7 | 240.9 ± 1.4 | 246.2 ± 1.1 | 251.1 ± 0.8 | 257.2 ± 0.6 | 263.7 ± 0.9 |
| Death | — | — | — | — | — | — | — |

Comment: “—”, no death.

The results showed that administration of strain CCFM8630 of Bifidobacterium adolescentis with a concentration of 3.0×10⁹ CFU/mL did not have significant influences on rats, there was no significant change on the body weight and no death. There were no obvious pathological symptoms in the appearance of the rats.

### Example 3: Strain CCFM8630 of Bifidobacterium adolescentis has Good Recovery Effect on Tissue Damages of Liver, Duodenum and so on in Rats with Metabolic Syndrome

48 healthy male SD rats with weight from 180 to 200 g were chosen and acclimated for 1 week. The rats were divided into 6 groups randomly: non-specific control group (NC), high-fat high-starch (HFHS) diet model control group, simvastatin control group (SC), rosiglitazone hydrochloride control group (RH), strain CCFM8630 of Bifidobacterium adolescentis intervention group (CCFM8630), Bifidobacterium animalis BB12 control group (BB12), 8 rats per group. The rats were administered with the bacteria suspension (3.0×10⁹ CFU/mL, in 2% sucrose solution) by intragastric gavage. Grouping and treatment method were shown in Table 5.

TABLE 5
Grouping and treatment method of the experiment

| | Number of | Treatment | | Treatment Method: administrated by |
|---|---|---|---|---|
| Group | rats/Group | Duration | Feed | intragastric gavage daily |
| NC | 8 | 12 Weeks | Normal feed | 2 ml of 2% sucrose solution |
| HFHS | 8 | 12 Weeks | High-fat high-starch | 2 ml of 2% sucrose solution |
| | | | feed | |
| SC | 8 | 12 Weeks | High-fat high-starch | 2 ml of 2% sucrose solution containing |
| | | | feed | 3 mg/kg/BW/d of simvastatin |
| RH | 8 | 12 Weeks | High-fat high-starch | 2 ml of 2% sucrose solution containing |
| | | | feed | 10 mg/kg/BW/d of rosiglitazone hydrochloride |
| CCFM8630 | 8 | 12 Weeks | High-fat high-starch | 2 ml of 2% sucrose solution containing |
| | | | feed | 3.0 × 10⁹ CFU/mL of CCFM8630 |
| BB12 | 8 | 12 Weeks | High-fat high-starch | 2 ml of 2% sucrose solution containing |
| | | | feed | 3.0 × 10⁹ CFU/mL of BB12 of |

At the end of the experiment, the rats were fasted (with access to water) for 12 hours. After administering 10% chloral hydrate by peritoneal injection, the rats were anesthetized, the blood samples were collected from the hearts, and the rats were sacrificed by cervical dislocation. The blood samples were centrifuged at a speed of 3000×g at 4° C. for 10 minutes. The supernatant was collected and frozen at −80° C. for later use. Liver, duodenum and so on were collected and quickly put into ice-cold physiological saline to wash away the blood, followed by fixation in paraformaldehyde. In addition, small intestine was collected and immediately frozen in liquid nitrogen.

Intestine, duodenum and so on were taken and prepared as paraffin sections, followed by HE staining. Morphology of the tissues were observed and imaged under optical microscope for pathological evaluation. The results were shown in FIGS. 2 and 3. The HE staining was performed by the following steps.

(1) Fixation: the tissue samples were washed with physiological saline and immediately put into neutral paraformaldehyde solution (4%) for fixation, and the duration of fixation was generally within 72 hours.

(2) Washing: the tissue samples were washed with running water or immersed in water for a few hours or overnight.

(3) Dehydration: the tissue samples were dehydrated by successively immersing in ethanol solutions of 70%, 80% and 90%, each for 30 minutes, and then immersing in 95% ethanol solution once for 20 minutes, immersing in 100% ethanol solution twice, each time for 10 minutes.

(4) Transparency: the tissue samples were immersed in a mixture of ½ absolute ethanol and ½ xylene for 10 minutes, xylene I for 10 minutes, and xylene II for 10 minutes (until the samples became transparent).

(5) Waxing: the tissue samples were placed in paraffin (at 62° C.) for 2 hours.

(6) Embedding: the largest side of the sample was placed in the bottom so that the sections have the largest tissue surface.

(7) Cutting: the wax blocks were cut by a manually operating microtome into slices with a thickness of 5 μm.

(8) Floatation and adhesion of sections (slice-salvaging): a water bath was used and the water was maintained at 42° C.; sections were placed onto the water surface smoothly.

(9) Drying: slides and slide rack were put into a 55° C. drying oven for about 2 hours until the wax melted.

(10) Hydration: slides were immersed in xylene I and II for 10 minutes respectively for dewaxing, and then immersed in ethanol solutions of 100%, 95%, 90%, 80% and 70% for 5 minutes respectively, and then immersed in distilled water for 3 minutes.

(11) Primary stain: the slides were put into hematoxylin solution and stained for about 20 seconds.

(12) Washing: the slides were washed with tap water for about 15 minutes until the slices became blue. Pay attention to the water flow to avoid the sections detaching from the slide.

(13) Differentiation: the slides were put into ethanol solution with 1% hydrochloric acid for 7 seconds until the slices turned red (the color became light).

(14) Rinsing: the slides were washed with tap water for 15 to 20 minutes until the color recovered blue.

(15) Re-stain: the slides were immersed in eosin solution and immediately taken out for dehydration.

(16) Dehydration: the slides were immersed in 95% ethanol solution I, 95% ethanol solution II and 70% ethanol solution successively, followed by immersing in 80% ethanol solution for 50 seconds and absolute ethanol for 2 minutes.

(17) Transparency: the slides were immersed in ½ of absolute ethanol and ½ of xylene for 1 minute, xylene I for 2 minutes and xylene II for 2 minutes, respectively.

(18) Sealing: after the treatment of xylene, the neutral balsam was used as mounting medium, which could be diluted to appropriate consistency with xylene.

FIG. 2 showed that high-fat high-starch diet caused hepatocyte microvesicular steatosis, and a number of rats have infiltration of inflammatory cell and hyperplasia of fibrous tissue. In high-fat high-starch diet model control group (HFHS), there was significant hyperplasia of fibrous tissue in liver tissue and morphologic features of early fibrosis. In strain CCFM8630 of Bifidobacterium adolescentis intervention group (CCFM8630), administration of strain CCFM8630 of Bifidobacterium adolescentis by intragastric gavage significantly improved the lesions above, and the effects were significantly better than that of group BB12. FIG. 3 showed that under optical microscope, lesions of duodenum were villi broadening, interstitial edema, increase of inflammatory cells and increase of interstitial macrophages in a few cases. In strain CCFM8630 of Bifidobacterium adolescentis intervention group (CCFM8630), administration of strain CCFM8630 of Bifidobacterium adolescentis by intragastric gavage improved the lesions above, and the effects were significantly better than that of group BB12.

### Example 4: Strain CCFM8630 of Bifidobacterium adolescentis has Recovery Effect on Intestinal Flora Imbalance Caused by High-Fat High-Starch Diet

Grouping, molding and treatment processes using SD rats were the same as described in Example 3. Before the end of the experiment, fresh feces of the rats were taken and metagenome samples were extracted. A second-generation sequencer was used for sequencing and the microbial community structure was analyzed.

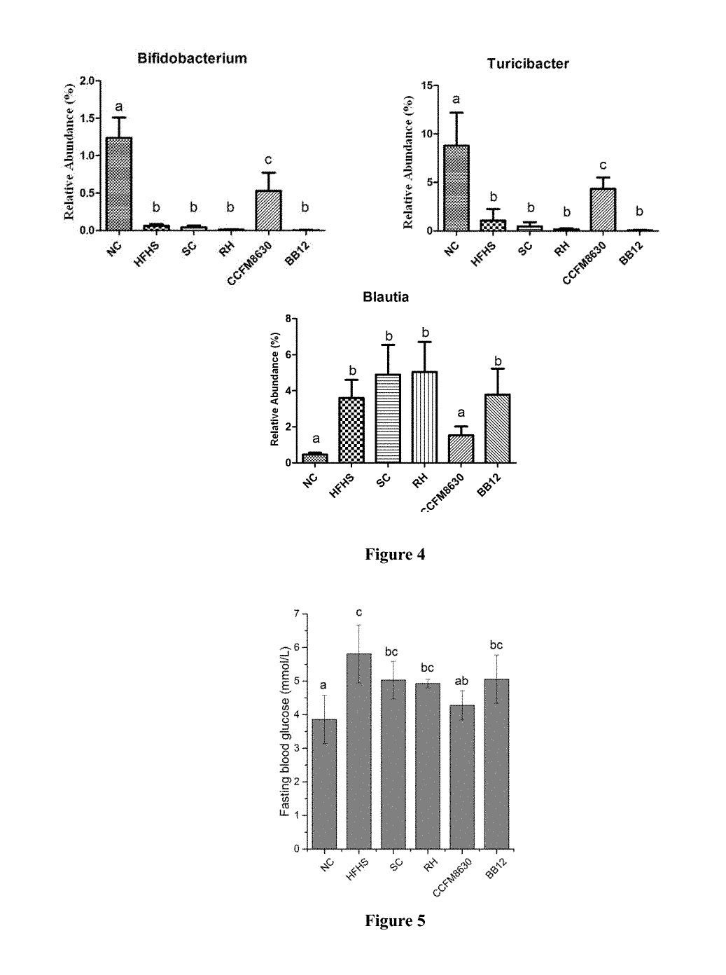

The experiment results were shown in FIG. 4. In feces of high-fat high-starch diet model control group (HFHS), relative abundances of intestinal microbes Bifidobacterium genus and Turicibacter genus significantly decreased. In strain CCFM8630 of Bifidobacterium adolescentis intervention group (CCFM8630), intake of strain CCFM8630 of Bifidobacterium adolescentis leaded to a significant recovery of the relative abundances of these two genera, while drugs and BB12 did not show recovery effect on the abundances of these two genera intestinal microbes. This also indicated that strain CCFM8630 of Bifidobacterium adolescentis can not only colonize in intestine, but also improve proportions of other species of Bifidobacterium genus in the intestine. In addition, in rat feces of the high-fat high-starch diet model control group, the relative abundance of intestinal microbes of Blautia genus significantly increased. In strain CCFM8630 of Bifidobacterium adolescentis intervention group, intake of strain CCFM8630 of Bifidobacterium adolescentis regulated the abundance of Blautia genus back to normal level, and the effects were better than that of the drugs and BB12.

### Example 5: Strain CCFM8630 of Bifidobacterium adolescentis Reduced (Fasting) Blood Glucose Level of Rats with Metabolic Syndrome

Grouping, molding and treatment processes using SD rats were the same as described in Example 3.

At the end of the experiment the rats were fasted (with access to water) for 12 hours and fasting blood glucose level of the rats was tested. The results were shown in FIG. 5.

In high-fat high-starch diet model control group (HFHS), fasting blood glucose level of rats significantly increased. In strain CCFM8630 of Bifidobacterium adolescentis intervention group (CCFM8630), administration of strain CCFM8630 of Bifidobacterium adolescentis by intragastric gavage significantly decreased fasting blood glucose level of model rats, approximately to non-specific control group, and its ability to decrease fasting blood glucose level of rat is better than that of rosiglitazone hydrochloride control group (RH) and Bifidobacterium animalis BB12 control group (BB12).

### Example 6: Strain CCFM8630 of Bifidobacterium adolescentis Increased Glucose Tolerance of Rat with Metabolic Syndrome

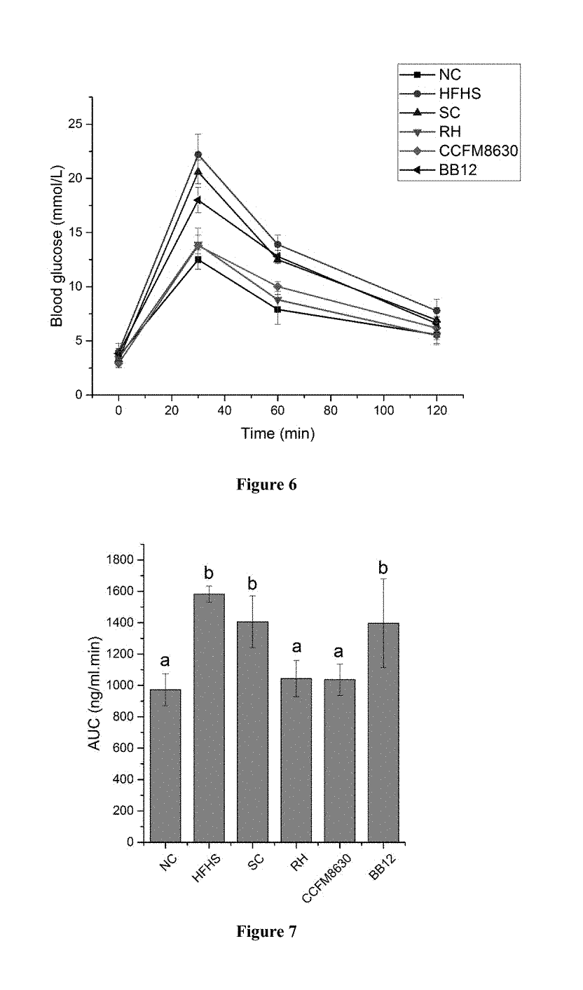

Grouping, molding and treatment processes using SD rats were the same as described in Example 3. At the end of the experiment, the rats were fasted (with access to water) for 12 hours. Glucose solution (2 g/kg) was injected by intraperitoneal injection and the blood glucose level was measured at 0, 30, 60 and 120 minutes. The experiment results were shown in FIGS. 6 and 7.

As shown in FIG. 6, glucose tolerance of rats in high-fat high-starch diet model control group (HFHS) was poor. After administration of glucose by intragastric gavage, blood glucose level rose significantly and decreased slowly. As shown in FIG. 7, in strain CCFM8630 of Bifidobacterium adolescentis intervention group (CCFM8630), administration of strain CCFM8630 of Bifidobacterium adolescentis by intragastric gavage significantly decreased AUC_{glucose} area, and there was no significant difference comparing with that of rosiglitazone hydrochloride control group (RH) and non-specific control group (NC). This indicated that strain CCFM8630 of Bifidobacterium adolescentis significantly improves oral glucose tolerance, and the effect was better than that of Bifidobacterium animalis BB12. These results were consistent with that of the blood glucose indexes, indicating that strain CCFM8630 of Bifidobacterium adolescentis further decreased blood glucose level by increasing glucose tolerance.

### Example 7: Strain CCFM8630 of Bifidobacterium adolescentis Decreased Total Cholesterol (TC) Level in Serum of Rat with Metabolic Syndrome

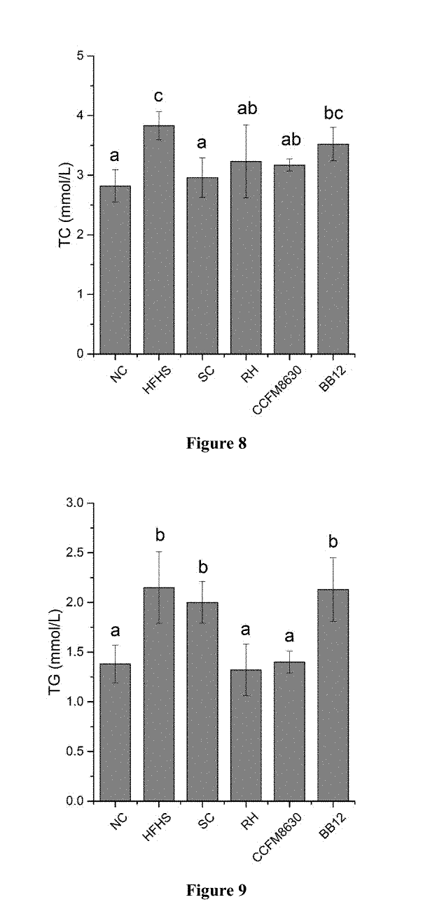

Grouping, molding and treatment processes using SD rats were the same as described in Example 3. At the end of the experiment, the rats were fasted (with access to water) for 12 hours. After administering 10% chloral hydrate by peritoneal injection for anesthetizing, blood sample was collected from the heart, and the rats were sacrificed by cervical dislocation. The blood samples were centrifuged at a speed of 3000×g at 4° C. for 10 minutes, and the supernatant was collected. The total cholesterol (TC) in the blood was measured according to the protocol of the detection kit. The experiment results were shown in FIG. 8.

As shown in FIG. 8, total cholesterol in serum of rats in high-fat high-starch diet model control group (HFHS) significantly increased. In strain CCFM8630 of Bifidobacterium adolescentis intervention group (CCFM8630), administration of strain CCFM8630 of Bifidobacterium adolescentis by intragastric gavage decreased level of total cholesterol in serum.

### Example 8: Strain CCFM8630 of Bifidobacterium adolescentis Decreased Triglyceride (TG) Level in Serum of Metabolic Syndrome Rat

Grouping, molding and treatment processes using SD rats were the same as described in Example 3. At the end of the experiment, the rats were fasted (with access to water) for 12 hours. After administering 10% chloral hydrate by peritoneal injection for anesthetizing, blood sample was collected from the heart, and the rats were sacrificed by cervical dislocation. The blood samples were centrifuged at a speed of 3000×g at 4° C. for 10 minutes, and the supernatant was collected. The triglyceride (TG) level in the blood was measured according to the protocol of the detection kit. The experiment results were shown in FIG. 9.

As shown in the experiment results, comparing with non-specific control group (NC), triglyceride level in serum of rats in high-fat high-starch diet model control group significantly increased. In strain CCFM8630 of Bifidobacterium adolescentis intervention group, administration of strain CCFM8630 of Bifidobacterium adolescentis by intragastric gavage decreased triglyceride level in serum, and the effect was equivalent to that of rosiglitazone hydrochloride control group. The administration of BB12 by intragastric gavage did not show significant effect.

### Example 9: Strain CCFM8630 of Bifidobacterium adolescentis Affected 5-HT and Testosterone Levels in Serum of Rat with Metabolic Syndrome

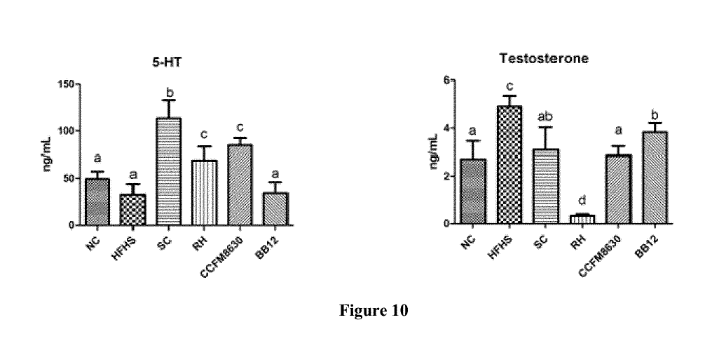

Grouping, molding and treatment processes using SD rats were the same as described in Example 3. At the end of the experiment, the rats were fasted (with access to water) for 12 hours. After administering 10% chloral hydrate by peritoneal injection for anesthetizing, blood sample was collected from the heart, and the rats were sacrificed by cervical dislocation. The blood samples were centrifuged at a speed of 3000×g at 4° C. for 10 minutes, and the supernatant was collected. The 5-HT and testosterone levels in the blood were measured according to the protocol of the detection kit. The experiment results were shown in FIG. 10.

As shown in the experiment results, strain CCFM8630 of Bifidobacterium adolescentis significantly increased 5-HT level in serum of rats, while BB12 has no significant improvement on 5-HT level. Comparing with non-specific control group (NC), testosterone level in serum of rats in high-fat high-starch diet model control group (HFHS) significantly increased. In strain CCFM8630 of Bifidobacterium adolescentis intervention group (CCFM8630), administration of strain CCFM8630 of Bifidobacterium adolescentis by intragastric gavage reduced the testosterone level in serum back to normal.

What is claimed is:

1. A composition comprising the strain CCFM8630 of Bifidobacterium adolescentis, which is deposited at China General Microbiological Culture Collection Center (CGMCC) with an accession number CGMCC 14395.

2. The composition according to claim 1, which is in the form of health food or pharmaceutical preparation.

3. The composition according to claim 2, wherein the health food is a microbial agent or a fermented food.

4. The composition according to claim 4, wherein the microbial agent comprises viable bacteria which is at least 10⁶ CFU/g of the strain CCFM8630 of Bifidobacterium adolescentis.

5. The composition according to claim 3, wherein the fermented food is produced by using the strain CCFM8630 of Bifidobacterium adolescentis as a starter culture.

6. The composition according to claim 3, wherein the fermented food is a fermented dairy product, a fermented bean product or a fermented fruit and vegetable product.

7. The composition according to claim 2, wherein the pharmaceutical preparation comprises an effective amount of the strain CCFM8630 of Bifidobacterium adolescentis and a pharmaceutically acceptable adjuvant.

8. A method of improving metabolic syndrome, modulating intestinal flora, improving irritable bowel syndrome, modulating brain-gut axis, relieving anxiety and/or depression, comprising administering a strain CCFM8630 of Bifidobacterium adolescentis which is deposited at China General Microbiological Culture Collection Center (CGMCC) with an accession number CGMCC 14395, to a subject in need thereof.

9. The method according to claim 8, wherein the improving of metabolic syndrome is to relieve the symptoms of hyperglycemia and hyperlipidemia, inflammation of liver and duodenum, and liver fibrosis; the modulating of intestinal flora is to normalize abnormal abundances of Blautia genus and Turicibacter genus in the intestinal flora; and the modulating of brain-gut axis and the relieving of anxiety and/or depression is to increase 5-hydroxytryptamine level in peripheral blood.

10. A method for preparing the composition according to claim 1, comprising: inoculating the strain CCFM8630 of Bifidobacterium adolescentis to a modified MRS medium at an inoculum size of 2 to 4 wt %, culturing for 24 to 48h at a temperature between 35 and 39° C. under anaerobic condition, collecting bacteria, resuspending the bacteria with a protectant to a bacterial density of 10¹⁰ CFU/mL, culturing the suspension at 37° C. for 50 to 70 minutes under anaerobic condition, and drying the resulting culture.

11. The method according to claim 10, wherein the modified MRS medium (mMRS) is a MRS medium containing 0.05% of L-cysteine hydrochloride; the protectant is a aqueous solution containing 100 g/L to 150 g/L of nonfat milk powder, 100 g/L to 150 g/L of maltodextrin, and 140 g/L to 160 g/L of trehalose; the drying is vacuum freeze-drying after pre-freezing at −15 to −20° C. for 8 to 14h.
